(19) United States
(12) Patent Application Publication
(10) Pub. No.: US 2025/0009276 A1
(43) Pub. Date: Jan. 9, 2025

(54) SYSTEM, DEVICE AND METHOD FOR DETERMINING LOCATION OF ARRHYTHMOGENIC FOCI

(71) Applicant: Riccardo Cappato, Milan (IT)

(72) Inventor: Riccardo Cappato, Milan (IT)

(21) Appl. No.: 18/895,067

(22) Filed: Sep. 24, 2024

Related U.S. Application Data
(63) Continuation-in-part of application No. 17/505,263, filed on Oct. 19, 2021.

(51) Int. Cl. A61B 5/364; A61B 5/00; A61B 5/287; A61F 2/95

(52) U.S. Cl. CPC A61B 5/364; A61B 5/0006; A61B 5/0031; A61B 5/287; A61B 5/6862; A61F 2/95; A61B 2560/0214

(57) ABSTRACT

A locator assembly (1400) for determining a location of arrhythmogenic foci (632) in or near a heart (1401), includes a device body (1412), a plurality of electrodes (1402), a component device (1480), such as a subcutaneous device and/or an extracorporeal device, and at least one of a communicator (1404), a controller (1406) and a power source (1410) that is incorporated within the component device (1480). The device body (1412) is provided in the form of an expandable stent that is configured to be inserted into and engage the heart (1401). The electrodes (1402) are coupled to the device body (1412). The electrodes (1402) are configured to sense electrical signals from the heart (1401) to determine the location of the arrhythmogenic foci (632). The component device (1480) is positioned spaced apart from the device body (1412). The at least one of the communicator (1404), the controller (1406), and the power source (1410) is configured to wirelessly communicate with the electrodes (1402).

### RELATED APPLICATION

This application is a continuation-in-part application of and claims priority on U.S. application Ser. No. 17/505,263, filed on Oct. 19, 2021, and entitled “SYSTEM, DEVICE, AND METHOD FOR DETERMINING LOCATION OF ARRHYTHMOGENIC FOCI”. As far as permitted, the contents of U.S. application Ser. No. 17/505,263 are incorporated in their entirety herein by reference.

### BACKGROUND

Atrial fibrillation is an irregular and sometimes rapid heart rate that can increase the risk of stroke, heart failure, and other heart-related complications. During atrial fibrillation, the heart's two upper chambers (the atria) beat chaotically and irregularly—out of coordination with the heart's two lower chambers (the ventricles). Atrial fibrillation symptoms often include heart palpitations, shortness of breath, and weakness. Although atrial fibrillation usually is not life-threatening, it is a severe medical condition that sometimes requires treatment. Atrial fibrillation can originate from focal sources (referred to herein as “arrhythmogenic foci”) in the atria or other locations in and around the heart.

Catheter ablation of atrial fibrillation is currently performed using an anatomically-based approach to the atrial substrate. Previous models hypothesize that most clinical atrial fibrillation episodes originate inside the pulmonary veins. Eligible patients for atrial fibrillation ablation are not representative of the typical patient with atrial fibrillation (e.g., eligible patients for atrial fibrillation ablation on average are ten years younger, commonly with fewer co-morbidities). As a result, catheter-based pulmonary vein isolation is unlikely to be an effective strategy to cure atrial fibrillation in the overall population of atrial fibrillation patients.

The anatomically-based approach to the atrial substrate surrogates the ability of clinicians to provide relevant electrophysiological information during clinical episodes of atrial fibrillation. Problems with the anatomically-based approach include (1) recurrent conduction across the isolating ablation lesions deployed at the pulmonary vein orifice/antrum, (2) precipitation of atrial fibrillation events from sites other than the pulmonary vein. Recurrence of atrial fibrillation events includes many patients among those in whom pulmonary vein isolation fails to control recurrences of atrial fibrillation. In all patients with previously successful pulmonary vein isolation, recurrent episodes still exist.

Mapping areas alternative to pulmonary veins that generate extra beats precipitating atrial fibrillation episodes is currently precluded by the inability to monitor real-time precipitating episodes. Other approaches, such as a surrogate strategy to real-time mapping, are represented by catecholamine-induced atrial fibrillation during ablation procedures. However, the surrogate strategy is not standardized, is time-consuming and ineffective (drug-induced atrial fibrillation does not represent spontaneous atrial fibrillation). Importantly, a major problem is the ability to accurately determine the precise location of the arrhythmogenic foci that is causing atrial fibrillation in the individual patient.

### SUMMARY

The present invention is directed toward a locator assembly for determining a location of arrhythmogenic foci in or near a heart within a body of a patient. In various embodiments, the locator assembly includes a device body, a plurality of electrodes, a component device, and at least one of a communicator, a controller and a power source that is incorporated within the component device. The device body is provided in the form of an expandable stent that is configured to be inserted into and engage the heart. The plurality of electrodes are coupled to the device body. The plurality of electrodes are configured to sense electrical signals from the heart to determine the location of the arrhythmogenic foci within the body of the patient. The component device is positioned spaced apart from the device body. When included, the communicator is configured to receive data regarding the sensed electrical signals from the plurality of electrodes and to transmit the data to an external device, the controller is configured to control operation of the plurality of electrodes, and the power source is configured to provide power to the plurality of electrodes. The at least one of the communicator, the controller, and the power source is configured to wirelessly communicate with the plurality of electrodes.

In some embodiments, the locator assembly further includes at least two of the communicator, the controller, and the power source that are incorporated within the component device, the at least two of the communicator, the controller, and the power source being configured to wirelessly communicate with the plurality of electrodes.

In certain embodiments, the locator assembly further includes each of the communicator, the controller, and the power source that are incorporated within the component device, each of the communicator, the controller, and the power source being configured to wirelessly communicate with the plurality of electrodes.

In many embodiments, the component device is a subcutaneous device that is positioned under skin of the patient.

In other embodiments, the component device is an extracorporeal device that is positioned adjacent to, but outside of the body of the patient.

In some embodiments, the locator assembly further includes a routing layer that interconnects the plurality of electrodes.

In various embodiments, the locator assembly further includes the power source that provides power to the plurality of electrodes.

In certain embodiments, the power source is rechargeable.

In other embodiments, the power source is self-charging.

In some embodiments, the locator assembly further includes an energy harvesting module that enables the power source to be self-charging.

In certain embodiments, the energy harvesting module includes (i) an inertial unit that is subject to external stresses that are applied to the device body when positioned inside the body of the patient, the external stresses causing oscillations of the inertial unit, and (ii) a translator that is configured to convert mechanical energy produced by the oscillations of the inertial unit into an oscillating electrical signal.

In some embodiments, the energy harvesting module further includes a power management circuit and an energy storage component, the power management circuit being configured to regulate the oscillating electrical signal in order to output a stabilized direct voltage or current for at least one of powering the plurality of electrodes and charging the energy storage component.

In certain embodiments, the device body is provided in the form of a self-expanding stent that is configured to be inserted into and engage the heart.

In some embodiments, the device body is configured to spontaneously move from a contracted state wherein the device body has a contracted diameter, to an expanded state wherein the device body has an expanded diameter that is greater than the contracted diameter.

In certain embodiments, a ratio of the expanded diameter to the contracted diameter is less than 20:1 and greater than 1:1.

In some embodiments, at least two of the plurality of electrodes are positioned circumferentially about the device body; and at least two of the plurality of electrodes are positioned longitudinally along the device body.

In certain embodiments, the plurality of electrodes includes a plurality of anodes and cathodes that form a plurality of bipoles.

In some embodiments, the plurality of electrodes includes an electrocardiogram electrode.

The present invention is further directed toward a locator system including a deployment catheter including a sheath; and the locator assembly as described above; wherein the device body is configured to spontaneously move from a contracted state to an expanded state; and wherein the device body is positioned within the sheath when the device body is inserted into the heart, the sheath being configured to maintain the device body in the contracted state.

In some embodiments, the device body spontaneously moves from the contracted state to the expanded state when the device body is removed from the sheath.

The present invention is also directed toward a method for determining a location of arrhythmogenic foci in or near a heart within a body of a patient, the method including the steps of coupling a plurality of electrodes to a device body to form at least a portion of a locator assembly, the device body including an expandable stent; inserting the device body within the heart; sensing electrical signals from the heart with the plurality of electrodes of the locator assembly; positioning a component device spaced apart from the device body; incorporating at least one of (i) a communicator that receives data regarding the sensed electrical signals from the plurality of electrodes and transmits the data to an external device, (ii) a controller that controls operation of the plurality of electrodes, and (iii) a power source that provides power to the plurality of electrodes, within the component device; wirelessly coupling the at least one of the communicator, the controller, and the power source with the plurality of electrodes; and determining the location of the arrhythmogenic foci within the body of the patient based at least in part on the electrical signals received from the heart by the plurality of electrodes.

This summary is an overview of some of the teachings of the present invention and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1A is a simplified perspective view of an embodiment of a locator assembly for locating arrhythmogenic foci in or near a heart and an external device, the locator assembly having features of the present invention;

FIG. 1B is a simplified illustration of the heart and an embodiment of the locator assembly for locating the arrhythmogenic foci in or near the heart, the locator assembly being positioned within a portion of the heart;

FIG. 2A is a simplified end view of an embodiment of the locator assembly, shown in a contracted state;

FIG. 2B is a simplified end view of an embodiment of the locator assembly, shown in an expanded state;

FIG. 3 is a simplified, partially transparent, perspective view of an embodiment of the locator assembly showing bipolar relationships between pairs of electrodes within the locator assembly;

FIG. 4A is a simplified end view of an embodiment of the locator assembly;

FIG. 4B is a simplified end view of yet another embodiment of the locator assembly;

FIG. 4C is a simplified end view of a portion of the embodiment of the locator assembly shown in FIG. 4B;

FIG. 4D is a simplified end view of another portion of the embodiment of the locator assembly shown in FIG. 4B;

FIG. 5A is a simplified, partially transparent view of a portion of the heart, an embodiment of the locator assembly and an embodiment of a deployment catheter, the locator assembly being shown in the contracted state;

FIG. 5B is a simplified, partially transparent view of a portion of the heart, an embodiment of the locator assembly and an embodiment of the deployment catheter, the locator assembly being shown in the expanded state;

FIG. 6A is a simplified, partially transparent view of a portion of the heart, another embodiment of the locator assembly, and another embodiment of the deployment catheter, the locator assembly being shown in an initial deployment position;

FIG. 6B is a simplified, partially transparent view of the portion of the heart, the locator assembly, and the deployment catheter illustrated in FIG. 6A, the locator assembly being shown in a partially deployed position;

FIG. 6C is a simplified, partially transparent view of the portion of the heart, the locator assembly, and the deployment catheter illustrated in FIG. 6A, the locator assembly being shown in a fully deployed position;

FIG. 6D is a simplified illustration of an embodiment of the locator assembly positioned within a portion of the heart, including a sinus rhythm foci, the arrhythmogenic foci, and a predicted foci in the heart;

FIG. 7 is a simplified diagram illustrating a sinus signal array, a first signal array, and a second signal array generated during one embodiment of a method for locating the arrhythmogenic foci in or near the heart;

FIG. 8 is a simplified diagram illustrating a superimposition of the first signal array and the second signal array over one another generated during one embodiment of the method for locating the arrhythmogenic foci, the superimposition being shown in an unmatched state;

FIG. 9 is a simplified diagram illustrating a superimposition of the first signal array and the second signal array over one another generated during an embodiment of the method for determining the location of the arrhythmogenic foci, the superimposition being shown in a matched state;

FIG. 10 is a flow chart outlining one embodiment of a method for determining the location of arrhythmogenic foci in the heart;

FIG. 11 is a flow chart outlining another embodiment of a method for determining the location of arrhythmogenic foci in the heart;

FIG. 12 is a flow chart outlining yet another embodiment of a method for determining the location of arrhythmogenic foci in the heart;

FIG. 13 is a simplified perspective view of still another embodiment of the locator assembly;

FIG. 14 is a simplified illustration of the heart and yet another embodiment of the locator assembly having a device body, with one or more components of the locator assembly being incorporated within a component device that is spaced apart from the device body; and

FIG. 15 is a simplified schematic illustration of a portion of still yet another embodiment of the locator assembly, which includes an energy harvesting module that is configured to self-charge electronic components of the locator assembly as the locator assembly is being utilized to perform a diagnostic procedure.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

The present invention is directed toward systems, devices, and related methods for determining the location of arrhythmogenic foci within a body of a patient. In many embodiments, the systems, devices, and related methods are configured to enable mapping of precipitating episodes of clinical atrial fibrillation during a patient's daily life. In particular, in various embodiments, a locator assembly 100 (also sometimes referred to as a “stent assembly” and/or a “stent”) can be implanted within the patient so that the locator assembly 100 can locate the origin of clinical atrial fibrillation in or near a heart 101 of the patient. In many embodiments, the locator assembly 100 is steadily positioned and/or inserted by an operator into the heart 101 of the patient to engage the heart 101 for purposes of determining an exact location of arrhythmogenic foci in or near the heart 101 of the individual patient. It is appreciated that the locator assembly 100 can be utilized to locate the origin of clinical atrial fibrillation and/or the location of the arrhythmogenic foci during any suitable time interval (such as up to 8 to 12 months in certain implementations) between the time of device implantation and any subsequent ablation procedure that utilizes such information and data gathered through use of the locator assembly 100. More specifically, during the noted time interval, spontaneous episodes of atrial fibrillation can be recorded and the activation sequence of precipitating beats of each single episode can be recorded and stored for reference at the time of any subsequent ablation procedure.

As used herein, the “heart” is understood to mean the heart including both atrial chambers, both ventricular chambers, the septum, the pulmonary veins, the coronary sinus, the fossa ovalis, the superior vena cava, the inferior vena cava, the muscular sleeves, the vascular walls, connected, electrically active tissues, and all other heart support structures in or near the heart.

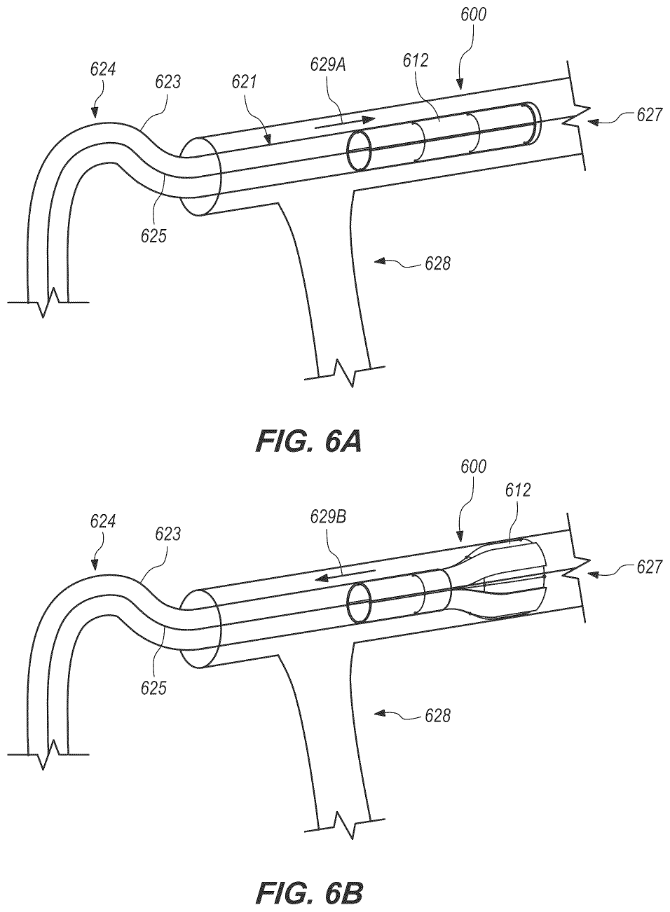

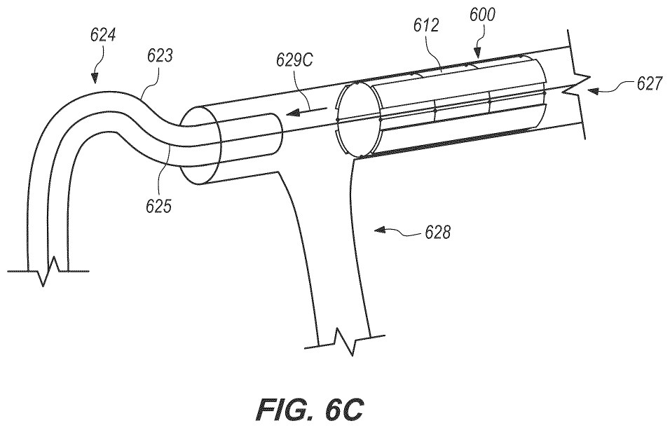

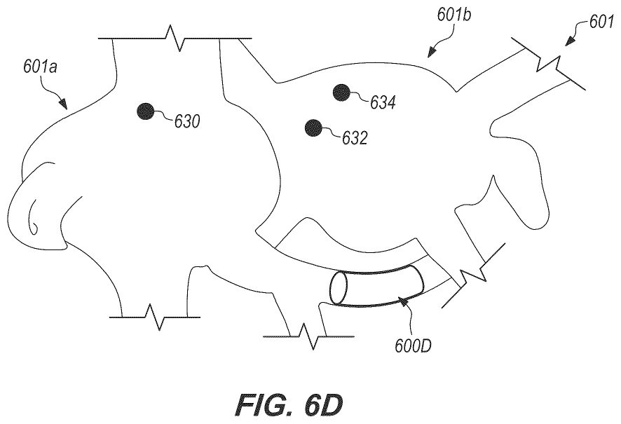

The locator assembly 100 can be used in the systems, devices, and methods described herein for determining a location of arrhythmogenic foci 632 (illustrated in FIG. 6D, for example) in or near the heart 101 of the patient. The systems, devices, and methods for determining the location of the arrhythmogenic foci 632 in or near the heart 101 described herein can vary.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention, as illustrated in the accompanying drawings. The same or similar nomenclature and/or reference indicators will be used throughout the drawings and the following detailed description to refer to the same or like parts.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is appreciated that such a development effort might be complex and time-consuming but would nevertheless be a routine undertaking of engineering for those of ordinary skill in the art having the benefit of this disclosure.

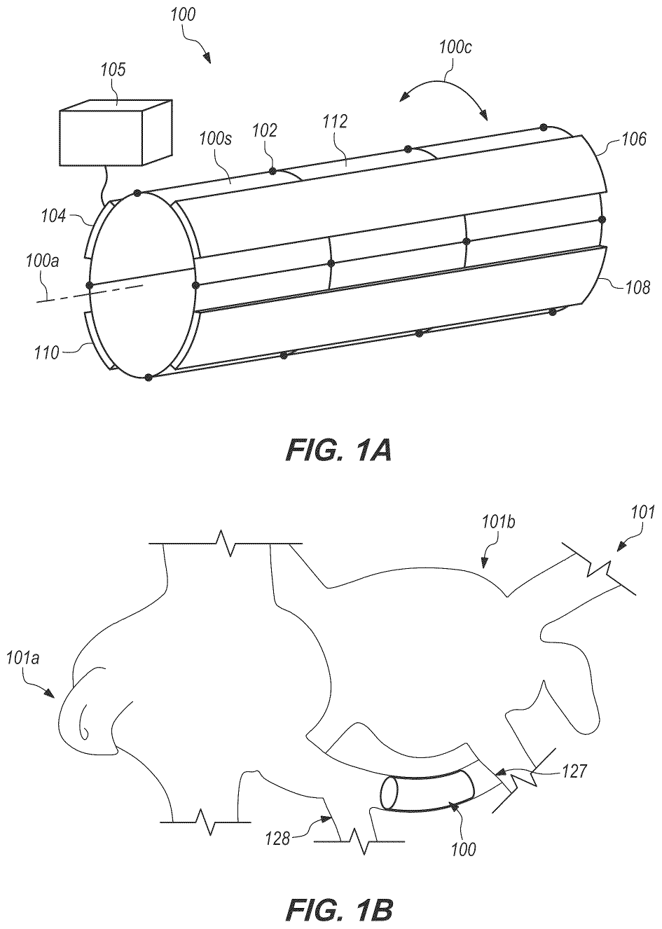

FIG. 1A is a simplified perspective view of an embodiment of a locator assembly 100 having features of the present invention that is utilized for locating arrhythmogenic foci 632 (illustrated in FIG. 6D) in or near the heart 101 (illustrated in FIG. 1B), and an external device 105 that is configured to communicate with the locator assembly 100. More particularly, as described, the locator assembly 100 is configured to generate and/or collect information and data for purposes of locating the arrhythmogenic foci 632 in or near the heart 101, and then such information and data is transmitted to the external device 105, such as by a wired connection or a wireless connection, for any necessary or desired processing.

As an overview, the present invention relates generally to implantable systems and methods, which incorporate the locator assembly 100, for performing intracardiac electrocardiogram (ECG) monitoring and arrhythmia trigger site identification. Subsequently, such site identification can be utilized for targeted tissue ablation to eliminate the sources of the arrythmia. In particular, the present invention provides a mapping stent and system which monitor and analyze intracardiac ECGs to identify areas causing atrial fibrillation and/or other arrhythmias for the purpose of targeting ablation and restoring long term normal sinus rhythm.

In various implementations, the locator assembly 100 is deliverable to and/or positionable within a portion of the heart 101 of a patient for performing a diagnostic procedure of locating the arrhythmogenic foci 632 in or near the heart 101. More particularly, in many implementations, the locator assembly 100 is steadily positioned by the operator into the heart 101 of the patient to engage the heart 101 for purposes of determining the exact location of the arrhythmogenic foci 632 in or near the heart 101 of the individual patient. For example, in certain implementations, the locator assembly 100 can be delivered to a coronary sinus 127 (illustrated in FIG. 1B) of the heart 101 (and near a vena cordis media 128 (illustrated in FIG. 1B) of the heart 101) for purposes of locating the arrhythmogenic foci 632. Additionally, or in the alternative, in other implementations, the locator assembly 100 can be delivered to and/or positioned within other portions of the heart 101, such as in or near a vein of Marshall of the heart 101, for purposes of locating the arrhythmogenic foci 632.

In some embodiments, where the locator assembly 100 may be implanted into blood vessels at different locations within the patient, such as for use in implementations where the locator assembly 100 may be positioned in both the coronary sinus 127 and the vein of Marshall, the locator assembly 100 can include anchors at opposing ends, with a connective cord running between.

By delivering, positioning and/or implanting the locator assembly 100 as so described, the locator assembly 100 can thus map precipitating episodes of clinical atrial fibrillation during the patient's daily life.

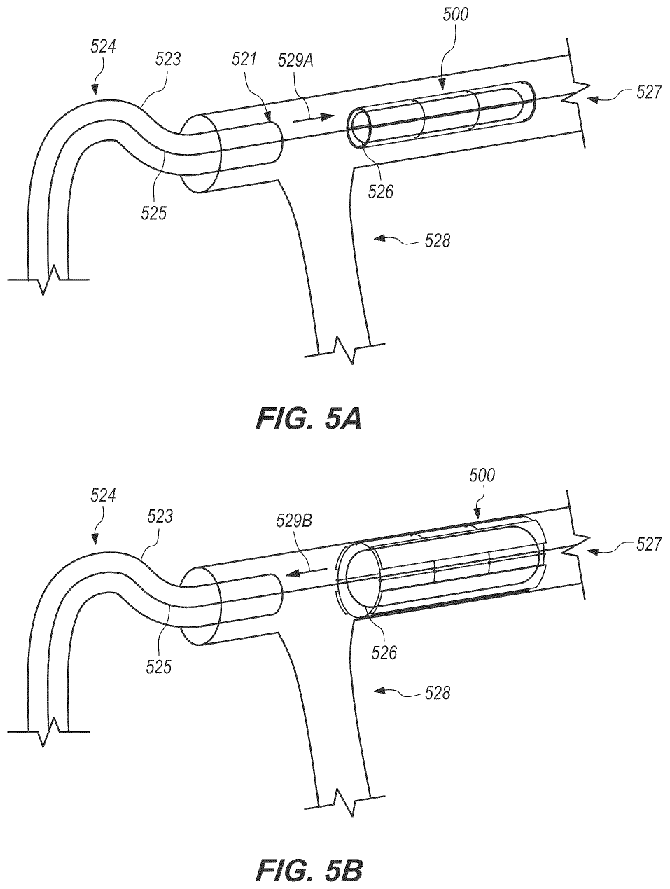

In various embodiments, the locator assembly 100 can be expandable to become anchored in a portion of the heart 101 of the patient. More particularly, in many embodiments, the locator assembly 100 can include a device body 112 that is steadily inserted into and expanded within the heart 101 of the patient. As such, in some embodiments, the locator assembly 100 and/or the device body 112 can include, incorporate and/or operate somewhat similarly to an expandable stent. In certain alternative embodiments, the locator assembly 101 and/or the device body 112 can be expandable through use of an inflatable balloon 526 (as shown, for example, in FIG. 5A), or the locator assembly 100 and/or the device body 112 can be self-expanding, with the device body 112 being configured to passively, spontaneously expand and become anchored in a portion of the heart 101 of the patient. Moreover, in some embodiments, at least one end of the device body 112 can incorporate an anchor, in any suitable form, to more effectively anchor the locator assembly 100 and/or the device body 112 at a desired location or target site within the heart 101 of the patient. For example, in one embodiment, the device body 112 can include a first (distal) anchor at or near a first (distal) end of the device body 112, and a second (proximal) anchor at or near a second (proximal) end of the device body 112, for purposes of anchoring the locator assembly 100 and/or the device body 112 at the desired location or target site within the heart 101 of the patient.

In many embodiments, the locator assembly 100 can be configured to provide cardiac telemetry monitoring and sampling of electrophysiological signals from the heart 101 of the patient. It is appreciated that, by providing the locator assembly 100 with telemetry capabilities, the locator assembly 100 can be more suitable for patients with asymptomatic, rare, or intermittent atrial fibrillation episodes.

In one embodiment, the locator assembly 100 can sample electrocardiogram (ECG) signals from the heart 101 of the patient periodically (in either even or uneven time increments) throughout a sampling period. In some implementations, the sampling period can be between one hour and one year. In other implementations, the sampling period can be less than one hour or greater than one year. The locator assembly 100 can capture an arrhythmia or arrhythmogenic foci 632 that may not be captured during a shorter sampling period by providing more extended sampling periods.

In various implementations, the locator assembly 100 can be placed within the patient permanently. In such implementations, the locator assembly 100 can include and/or incorporate an expandable stent to function long-term within the heart 101 of the patient. Alternatively, the locator assembly 100 can be removably positioned within the patient, such that the locator assembly 100 would likely be removed after the locator assembly 100 runs out of stored power, to replace or repair various components, or for any other suitable purpose.

As shown, the locator assembly 100 and/or the device body 112 has at least a longitudinal axis 100a, but may also have other axes. The locator assembly 100 and/or the device body 112 also has a circumference 100c that is measured about an outer surface 100s of the device body 112, such as in a direction substantially transverse to the longitudinal axis 100a.

In some embodiments, if the cross-section of the locator assembly 100 and/or the device body 112 is a perfect circle and the longitudinal axis 100a is perfectly centered through an end of the locator assembly 100, all positions on the circumference 100c are equidistant from the longitudinal axis 100a.

In various embodiments, the locator assembly 100 and its incorporated elements and components thereof can be rechargeable. In one embodiment, the locator assembly 100 can be wirelessly recharged while the locator assembly 100 is positioned within the patient. Alternatively, in other embodiments, the locator assembly 100 can be configured to incorporate self-charging capabilities, such that the locator assembly 100 can be recharged and/or self-charged while the locator assembly 100 is positioned within the patient.

The locator assembly 100 can vary depending on its design requirements. It is understood that the locator assembly 100 can include additional components, systems, subsystems, and elements other than those specifically shown and/or described herein. Additionally, or alternatively, the locator assembly 100 can omit one or more of the components, systems, subsystems, and elements that are specifically shown and/or described herein. In some embodiments, various components of the locator assembly 100 can be positioned in a different manner than what is specifically illustrated in FIG. 1A. In some embodiments, the locator assembly 100 can have the same or a somewhat similar design to a bare-metal stent, as one non-limiting, non-exclusive example.

Components of the locator assembly 100 can be configured to operate for a finite period or an average lifespan of the patient, if not longer. If necessary, some or all of the components and/or elements of the locator assembly 100 could potentially become immobilized during the extraction and/or replacement of the locator assembly 100.

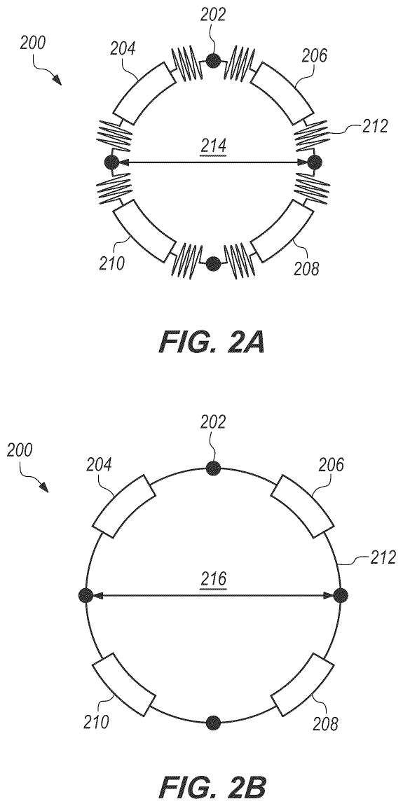

As illustrated in the embodiment shown in FIG. 1A, the locator assembly 100 can include the device body 112, and can further include a plurality of electrodes 102 (only one electrode is identified, with other electrodes shown as black dots in FIG. 1A, FIGS. 2A-2B, and FIGS. 3-4), a communicator 104, a controller 106, a routing layer 108, and a power source 110 (such as a battery), which can be coupled and/or secured to the device body 112. As used herein, the “components” of the locator assembly 100 can include the plurality of electrodes 102, the communicator 104, the controller 106, the routing layer 108, and the power source 110.

Alternatively, in other embodiments, the locator assembly 100 can again include the device body 112, but can be configured without one or more of the components noted herein being coupled and/or secured to the device body 112. For example, in certain non-exclusive alternative embodiments, the locator assembly 100 can be configured without one or more of the plurality of electrodes 102, the communicator 104, the controller 106, the routing layer 108 and/or the power source 110 being coupled and/or secured to the device body 112.

In some non-exclusive alternative embodiments, the locator assembly 100 can include the device body 112 with the plurality of electrodes 102 coupled and/or secured thereto, and one or more of the communicator 104, the controller 106, the routing layer 108 and the power source 110 can instead be included within a subcutaneous device (not shown in FIG. 1A) that can be positioned spaced apart from the device body 112, but can still be positioned within the body, such as under the skin, of the patient. In such embodiments, the subcutaneous device, and the components incorporated therewithin, can be positioned in such a manner as to still enable communication, such as wireless communication in many embodiments, between such components and the plurality of electrodes 102 that are coupled and/or secured to the device body 112.

In other embodiments, one or more of the communicator 104, the controller 106, the routing layer 108 and/or the power source 110 can instead be included within an extracorporeal device (not shown in FIG. 1A) that is positioned near and/or adjacent to the body of the patient, but not under the skin of the patient. Stated in another manner, in these embodiments, the extracorporeal device is positioned outside the body of the patient. In such embodiments, the extracorporeal device, and the components incorporated therewithin, can again be positioned in such a manner as to still enable communication, such as wireless communication in many embodiments, between such components and the plurality of electrodes 102 that are coupled and/or secured to the device body 112.

As referred to elsewhere herein, the subcutaneous device and/or the extracorporeal device which includes and/or incorporates at least one of the components of the locator assembly 100 spaced apart from the device body 112 can also be referred to generally as a “component device”.

Still alternatively, in another non-exclusive alternative embodiment, the locator assembly 100 can be configured without each of the plurality of electrodes 102, the communicator 104, the controller 106, the routing layer 108 and the power source 110. In such alternative embodiment, the locator assembly 100 can only include the device body 112 and can function simply as a stent within or near the heart 101 of the patient, or within any other vessel within the body of the patient. It is appreciated that such embodiment would not specifically include the capabilities for locating arrhythmogenic foci 632 in or near the heart 101, but would rather simply function as a stent for purposes of holding open whatever vessel of the body of the patient in which the locator assembly 100 is deployed.

In various embodiments, the locator assembly 100 can be configured for use by the patient while the patient receives a magnetic resonance imaging scan, or other imaging procedures. In other words, the locator assembly 100 can have shielding and/or resistance to varying types of external electromagnetic radiation. In some embodiments, the locator assembly 100 can be automatically activated and/or powered on. In certain embodiments, the locator assembly 100 can be manually activated and/or powered on by the patient or health care personnel.

As shown in FIG. 1A, the components of the locator assembly 100, such as the electrodes 102, the communicator 104, the controller 106, the routing layer 108, and the power source 110, can be radially spaced apart from one another about the circumference 100c of the device body 112. For example, in various non-exclusive embodiments, the components of the locator assembly 100 can be spaced apart by 1, 5, 10, 15, 20, 25, 30, 35, 40, 45, 60, 90, 120, or 180 degrees about the circumference 100c of the device body 112. In other embodiments, the components of the locator assembly 100 can be spaced apart by less than 1 degree or some other radial spacing other than those listed herein.

It is appreciated that the components of the locator assembly 100 can be positioned as provided above, even if the cross-sectional shape of the device body 112 is something other than a circle. The cross-sectional shape of the device body 112 can be any suitable shape. Non-limiting, non-exclusive examples of the cross-sectional shape of the device body 112 include circular-shaped, oval-shaped, egg-shaped, pentagonal-shaped, hexagonal-shaped, heptagonal-shaped, octagonal-shaped, decagonal-shaped, or any suitable shape. The cross-sectional shape of the device body 112 can have any number of sides and any type of curvature.

In some embodiments, the components of the locator assembly 100 can be spaced apart substantially equidistant from each other about the circumference 100c of the device body 112. The locator assembly 100 can include a plurality of platforms (not shown) configured to retain corresponding components of the locator assembly 100 about the circumference 100c of the device body 112. In certain embodiments, such as shown in FIG. 1A, the components of the locator assembly 100 can integrate platforms configured to enable coupling to the device body 112 of the locator assembly 100.

The electrodes 102 record and sense electrical signals (such as electrophysiological signals) sent from the heart 101 and nearby portions of the body. In some embodiments, the electrodes 102 can record the atrial activity and related electrical impulses. Thus, in certain embodiments, the plurality of electrodes 102 can be coupled to the device body 112 to receive and record electrical signals from the heart 101 so as to serve as a stable electrophysiological reference.

The type of electrodes 102 can vary depending on the design requirements of the locator assembly 100. In some embodiments, the electrodes 102 can be positioned in different configurations than what is specifically illustrated in FIG. 1A.

The electrodes 102 can include any suitable types of electrodes, including one or more electrocardiogram electrodes (as a non-limiting, non-exclusive example). The electrodes 102, when positioned in pairs, can form bipolar electrodes. The electrodes 102 can be coupled and decoupled from the device body 112 to repair or replace defective or otherwise inoperable electrodes 102 of the locator assembly 100. The locator assembly 100 can include any suitable number of electrodes 102. In some embodiments, such as the embodiment shown in FIG. 1A, the locator assembly 100 can include 16 electrodes 102. In other embodiments, the locator assembly 100 can include 2, 4, 6, 8, 10, 12, 14, 16, 18, 20, 22, 24, 26, 28, 30, or 32 electrodes 102. In certain embodiments, the locator assembly can 100 can include greater than 32 electrodes.

The electrodes 102 can be distributed about the circumference 100c of the device body 112 in a pattern, either in the longitudinal and/or circumferential directions or on any suitable portion of the locator assembly 100. About the circumference 100c of the device body 112, the electrodes 102 can be spaced apart by 10, 20, 30, 45, 60, 72, 90, 120, or 180 degrees. In other embodiments, the electrodes 102 can be positioned approximately 5, 15, 25, 35, 40, 50, 55, 65, 70, 75, 80, 85, 95, 100, 105, 110, 115, 125, 130, 135, 140, 145, 150, 155, 160, 165, 170, 175 or any other suitable spacing from one another along the circumference 100c of the device body 112.

In some embodiments, the electrodes 102 can be distributed in a somewhat circular, oval, cylindrical, or any suitable pattern about the device body 112. In one embodiment, the electrodes 102 can be evenly spaced apart from one another along the longitudinal axis 100a and/or about the circumference 100c of the device body 112. In alternative embodiments, the electrodes 102 can be spaced apart from one another along the longitudinal axis 100a and/or about the circumference 100c of the device body 112 in an uneven, asymmetrical, semi-random or random manner.

In many embodiments, the communicator 104 is used by the locator assembly 100 for wireless communication between the locator assembly 100 and the external device 105 (such as a computing device). In other words, the communicator 104 is configured to allow communication between the locator assembly 100 and the external device 105. More particularly, the communicator 104 can function as a transmitter and/or a receiver for purposes of enabling wireless communication between the locator assembly 100 and the external device 105. For example, data collected by the locator assembly 100, such as the electrical signals from the heart 101 that are sensed by the electrodes 102, can be sent wirelessly via the communicator 104 to the external device 105. Information, data and/or instructions for the locator assembly 100 can also be sent wirelessly from the remote device 105 to the locator assembly 100 via the communicator 104. Alternatively, the communicator 104 can allow for wired communication between the locator assembly 100 and the external device 105.

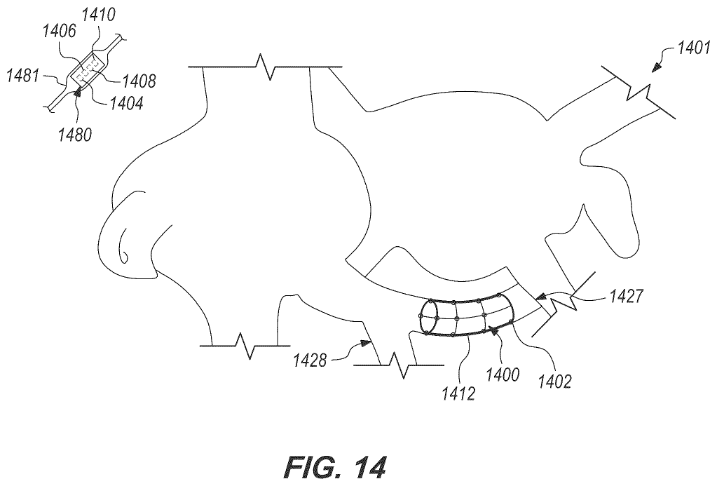

The type of communicator 104 and/or the positioning of the communicator 104 can vary depending on the design requirements of the locator assembly 100. The communicator 104 can include any suitable wireless communications device, such as a radio frequency, Bluetooth®, low energy antenna, and/or any suitable antenna as non-limiting, non-exclusive examples. The communicator 104 can also include any suitable wired communication device, such as wire antennas, dipole antennas, monopole antennas, loop antennas, transmission line antennas, etc. In some embodiments, the communicator 104 can be positioned differently than what is specifically illustrated in FIG. 1A. For example, in certain non-exclusive alternative embodiments, as illustrated and described in greater detail herein below, the communicator 104 can be coupled to and/or positioned substantially within a component device 1480 (illustrated in FIG. 14), which can be a subcutaneous device and/or an extracorporeal device, and which can be positioned spaced apart from the device body 112.

The external device 105 can communicate via the communicator 104 to enable (i) the transfer of data between the locator assembly 100 and the external device 105, (ii) the utilization of the memory of the external device 105 to increase processing speeds of the locator assembly 100, and/or (iii) the storage of data on the external device 105 following the transfer of the data from the locator assembly 100 to the external device 105. In some embodiments, the external device 105 can communicate with the communicator 104 to execute a set of processing instructions on the locator assembly 100. For example, the external device 105 can communicate via the communicator 104 to power the locator assembly 100 on or off.

The external device 105 can vary depending on the design requirements of the locator assembly 100. The connection between the communicator 104 and the external device 105 is merely demonstrative. The connection can indicate a wired and/or wireless connection between the locator assembly 100, the communicator 104, and/or the external device 105.

The controller 106 can control the other components of the locator assembly 100. The controller 106 can vary depending on the design requirements of the locator assembly 100. In some embodiments, the controller 106 can be positioned differently than what is specifically illustrated in FIG. 1A. For example, in certain non-exclusive alternative embodiments, as illustrated and described in greater detail herein below, the controller 106 can be coupled to and/or positioned substantially within the component device 1480, which can be a subcutaneous device and/or an extracorporeal device, and which can be positioned spaced apart from the device body 112.

The controller 106 can include (as non-limiting, non-exclusive examples) processors, microprocessors, diodes, capacitors, power storage elements, ASICs, sensors, image elements (such as CMOS, CCD imaging elements), amplifiers, A/D, and D/A converters, associated differential amplifiers, buffers, optical collectors, transducers including electro-mechanical transducers, piezoelectric actuators, light-emitting electronics which include LEDs, logic, memory, clock, and transistors including active matrix switching transistors, and combinations thereof. Components within electronic devices or devices are described herein and include those components described herein. A component can be one or more of any of the electronic devices described herein and/or may include a photodiode, LED, TUFT, electrode, semiconductor, other light-collecting/detecting components, transistor, contact pad capable of contacting a device component, thin-film devices, circuit elements, control elements, microprocessors, interconnects, contact pads, capacitors, resistors, inductors, a memory element, power storage element, antenna, logic element, buffer and/or other passive or active components. A component of the locator assembly 100 may be connected to one or more contact pads as known in the art, such as via metal evaporation, wire bonding, application of solids or conductive pastes, and the like. The processor within the controller 106 can process and store data from each of the plurality of electrodes 102.

In certain embodiments, the controller 106 can include a control circuit that continuously monitors the ECG of the patient via communication with the plurality of electrodes 102 that are sensing and/or recording electrical signals (such as electrophysiological signals) sent from the heart 101 and nearby portions of the body. The controller 106 can further include a fibrillation detection algorithm, which when fibrillation is detected, records the pre-fibrillation ECG and the fibrillation data for subsequent transmission, such as to the remote device 105 and/or the component device 1480.

The routing layer 108 routes the other components of the locator assembly 100 and/or the controller 106 to properly connect the components according to the design of the locator assembly 100 and/or the controller 106. For example, the routing layer 108 can interconnect the electrodes 102.

The routing layer 108 can vary depending on the design requirements of the locator assembly 100 and/or the controller 106. In some embodiments, the routing layer 108 can be positioned differently than what is specifically illustrated in FIG. 1A. For example, in certain non-exclusive alternative embodiments, as illustrated and described in greater detail herein below, the routing layer 108 can be coupled to and/or positioned substantially within the component device 1480, which can be a subcutaneous device and/or an extracorporeal device, and which can be positioned spaced apart from the device body 112.

The routing layer 108 can include wiring, substrates, and/or other circuitry that are encased in a non-conductive dielectric material.

The power source 110 stores power and provides power to the various other components of the locator assembly 100. The power source 110 can vary depending on the design requirements of the locator assembly 100. In some embodiments, the power source 110 can be positioned differently than what is specifically illustrated in FIG. 1A. For example, in certain non-exclusive alternative embodiments, as illustrated and described in greater detail herein below, the power source 110 can be coupled to and/or positioned substantially within the component device 1480, which can be a subcutaneous device and/or an extracorporeal device, and which can be positioned spaced apart from the device body 112.

The power source 110 can be any suitable power source for use within the locator assembly 100 and/or can provide power to the various other components of the locator assembly 100 in any suitable manner. In certain embodiments, the power source 110 can be a single-use/disposable battery, or it can be a rechargeable battery. Alternatively, in other embodiments, the power source 110 can encompass and/or include a self-charging design in which the power source 110 effectively charges itself as the locator assembly 100 is performing a diagnostic procedure. Non-limiting, non-exclusive examples of the power source 110 and/or battery that can be used within the locator assembly 100 include alkaline, lithium, lithium-ion, lithium-iron-phosphate, lithium silicon, magnesium, mercury, mercury-oxide, silver-oxide, silver-zinc, zinc-air, zinc-carbon, zinc-chloride, lead, lead-acid gel, nickel-cadmium, nickel oxyhydroxide, nickel-metal hydride, nickel-zinc, and Absolyte® batteries. The power source 110 can also be a solid-state battery. The power source 110 can be any suitable size and/or shape for use within the locator assembly 100, such as the partial-cylinder shape illustrated in the embodiment shown in FIG. 1A.

In some embodiments, the power source 110 can be configured to power the locator assembly 100 for five years or more. In certain embodiments, the power source 110 can be configured to power the locator assembly 100 for less than five years, such as six months, one year, two years, three years, four years, or another suitable time frame less than five years. In various embodiments, the power source 110 can be wirelessly recharged. In another embodiment, the power source 110 can include a capacitor. In still another embodiment, the power source 110 can have self-charging capabilities.

The device body 112 provides at least some structure for the locator assembly 100. The device body 112 can provide a substrate to secure various components of the locator assembly 100. The device body 112 can include a framework and/or a lattice structure for expansion and contraction. In some embodiments, when the framework in the device body 112 expands in circumference, a longitudinal length of the device body 112 does not expand. In other embodiments, when the framework in the device body 112 expands in circumference, the longitudinal length of the device body 112 can also expand.

In certain embodiments, when the framework in the device body 112 expands in circumference and/or longitudinal length, the electrodes 102, the communicator 104, the controller 106, the routing layer 108, and the power source 110 also expand in circumference and/or longitudinal length. In some embodiments, when the framework in the device body 112 contracts in circumference and/or longitudinal length, the electrodes 102, the communicator 104, the controller 106, the routing layer 108, and the power source 110 also contract in circumference and/or longitudinal length. The various components of the locator assembly 100 including the electrodes 102, the communicator 104, the controller 106, the routing layer 108, and the power source 110, can be formed from flexible and/or expandable materials.

As described in detail herein, in various embodiments, the device body 112 can expand and contract as needed to deploy and extract the locator assembly 100 within various regions of the heart 101 and body of the patient. Stated in another manner, the device body 112 of the locator assembly 100 is movable between a contracted state and an expanded state. In certain implementations, the locator assembly 100 and/or the device body 112 can be implanted and maintained in position within the heart 101 for long-term or permanent usage within the heart 101 for purposes of locating arrhythmogenic foci in or near the heart 101. In such implementations, when in the expanded state, the locator assembly 100 and/or the device body 112 can function as a stent to hold open portions of the heart 101 such as valves, veins, sinuses, etc. In some implementations, due to its ability to move between the contracted state and the expanded state, the locator assembly 100 and/or the device body 112 can be said to include, function as and/or can be referred to as an “expandable stent”.

The device body 112 can vary depending on the design requirements of the locator assembly 100. In some embodiments, the device body 112 can be configured differently than what is specifically illustrated in FIG. 1A. The device body 112 can be any suitable structure known in the art that only allows expansion and contraction in circumference. The cross-sectional shape of the device body 112 in the contracted state and the expanded state can vary. Non-limiting, non-exclusive examples of the cross-sectional shape of the device body 112 include circular-shaped, oval-shaped, egg-shaped, pentagonal-shaped, hexagonal-shaped, heptagonal-shaped, octagonal-shaped, decagonal-shaped, or any suitable shape.

FIG. 1B is a simplified illustration of the heart 101 and an embodiment of the locator assembly 100 positioned within the heart 101. The heart 101 includes a right atrium 101a and a left atrium 101b. As shown, the locator assembly 100 can be flexible to conform to portions of the heart 101 such as valves, veins, sinuses, etc. In particular, in the embodiment shown in FIG. 1B, the locator assembly 100 can be positioned in a coronary sinus 127 near a vena cordis media 128. However, it is understood that the locator assembly 100 can equally be positioned in other locations in or around the heart 101, such as in or near a vein of Marshall, or any other suitable location.

FIG. 2A is a simplified front elevation view of an embodiment of the locator assembly 200 being shown in the contracted state. As used herein, the “contracted state” is understood to mean the locator assembly 200 and/or the device body 212 is contracted or unexpanded. In the contracted state, the structures and/or components, including the electrodes 202, the communicator 204, the controller 206, the routing layer 208, and the power source 210 within the locator assembly 200, can be at least partially contracted. For example, in one embodiment of the locator assembly 200 shown in FIG. 2A, the device body 212 is in the contracted state when the framework in the device body 212 is contracted or unexpanded. For ease of understanding, the contracted state of the device body 212 in FIG. 2A is exaggerated to demonstrate the flexibility and/or contraction of the device body 212.

In the embodiment shown in FIG. 2A, the device body 212 is the only component shown to be in the contracted state. While in the contracted state, the locator assembly 200 and/or the device body 212 has a contracted diameter 214. In some embodiments, the contracted diameter 214 illustrated and described herein can be between approximately 0.01 mm and 20.00 mm. In various non-exclusive embodiments, the contracted diameter 214 can be approximately 0.01 mm, 0.1 mm, 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, 5 mm, 5.1 mm, 5.2 mm, 5.3 mm, 5.4 mm, 5.5 mm, 5.6 mm, 5.7 mm, 5.8 mm, 5.9 mm, 6 mm, 6.1 mm, 6.2 mm, 6.3 mm, 6.4 mm, 6.5 mm, 6.6 mm, 6.7 mm, 6.8 mm, 6.9 mm, 7 mm, 7.1 mm, 7.2 mm, 7.3 mm, 7.4 mm, 7.5 mm, 7.6 mm, 7.7 mm, 7.8 mm, 7.9 mm, 8 mm, 8.1 mm, 8.2 mm, 8.3 mm, 8.4 mm, 8.5 mm, 8.6 mm, 8.7 mm, 8.8 mm, 8.9 mm, 9 mm, 9.1 mm, 9.2 mm, 9.3 mm, 9.4 mm, 9.5 mm, 9.6 mm, 9.7 mm, 9.8 mm, 9.9 mm, 10 mm, 10.1 mm, 10.2 mm, 10.3 mm, 10.4 mm, 10.5 mm, 10.6 mm, 10.7 mm, 10.8 mm, 10.9 mm, 11 mm, 11.1 mm, 11.2 mm, 11.3 mm, 11.4 mm, 11.5 mm, 11.6 mm, 11.7 mm, 11.8 mm, 11.9 mm, 12 mm, 12.1 mm, 12.2 mm, 12.3 mm, 12.4 mm, 12.5 mm, 12.6 mm, 12.7 mm, 12.8 mm, 12.9 mm, 13 mm, 13.1 mm, 13.2 mm, 13.3 mm, 13.4 mm, 13.5 mm, 13.6 mm, 13.7 mm, 13.8 mm, 13.9 mm, 14 mm, 14.1 mm, 14.2 mm, 14.3 mm, 14.4 mm, 14.5 mm, 14.6 mm, 14.7 mm, 14.8 mm, 14.9 mm, 15 mm, 15.1 mm, 15.2 mm, 15.3 mm, 15.4 mm, 15.5 mm, 15.6 mm, 15.7 mm, 15.8 mm, 15.9 mm, 16 mm, 16.1 mm, 16.2 mm, 16.3 mm, 16.4 mm, 16.5 mm, 16.6 mm, 16.7 mm, 16.8 mm, 16.9 mm, 17 mm, 17.1 mm, 17.2 mm, 17.3 mm, 17.4 mm, 17.5 mm, 17.6 mm, 17.7 mm, 17.8 mm, 17.9 mm, 18 mm, 18.1 mm, 18.2 mm, 18.3 mm, 18.4 mm, 18.5 mm, 18.6 mm, 18.7 mm, 18.8 mm, 18.9 mm, 19 mm, 19.1 mm, 19.2 mm, 19.3 mm, 19.4 mm, 19.5 mm, 19.6 mm, 19.7 mm, 19.8 mm, 19.9 mm, or 20 mm. In other embodiments, the contracted diameter 214 can be less than approximately 0.01 mm or greater than approximately 20.00 mm.

FIG. 2B is a simplified front elevation view of an embodiment of the locator assembly 200 being shown in the expanded state. In particular, FIG. 2B again illustrates that the locator assembly 200 includes the electrodes 202, the communicator 204, the controller 206, the routing layer 208, the power source 210 and the device body 212, but the locator assembly 200 has now been moved to the expanded state, such that the device body 212 has been expanded relative to the condition when the device body 212 is in the contracted state.

As used herein, the “expanded state” is understood to mean the locator assembly 200 and/or the device body 212 is expanded outwardly from the contracted state so that the locator assembly 200 and/or the device body 212 has an increased circumference and/or an increased diameter relative to the contracted state. The locator assembly 200 is movable between the contracted state and the expanded state.

While in the expanded state, the locator assembly 200 and/or the device body 212 has an expanded diameter 216 that is greater than the contracted diameter 214 (illustrated in FIG. 2A). In some embodiments, the expanded diameter 216 illustrated and described herein can be between approximately 0.01 mm and 20.00 mm. In various non-exclusive embodiments, the expanded diameter 216 can be approximately 0.01 mm, 0.1 mm, 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, 5 mm, 5.1 mm, 5.2 mm, 5.3 mm, 5.4 mm, 5.5 mm, 5.6 mm, 5.7 mm, 5.8 mm, 5.9 mm, 6 mm, 6.1 mm, 6.2 mm, 6.3 mm, 6.4 mm, 6.5 mm, 6.6 mm, 6.7 mm, 6.8 mm, 6.9 mm, 7 mm, 7.1 mm, 7.2 mm, 7.3 mm, 7.4 mm, 7.5 mm, 7.6 mm, 7.7 mm, 7.8 mm, 7.9 mm, 8 mm, 8.1 mm, 8.2 mm, 8.3 mm, 8.4 mm, 8.5 mm, 8.6 mm, 8.7 mm, 8.8 mm, 8.9 mm, 9 mm, 9.1 mm, 9.2 mm, 9.3 mm, 9.4 mm, 9.5 mm, 9.6 mm, 9.7 mm, 9.8 mm, 9.9 mm, 10 mm, 10.1 mm, 10.2 mm, 10.3 mm, 10.4 mm, 10.5 mm, 10.6 mm, 10.7 mm, 10.8 mm, 10.9 mm, 11 mm, 11.1 mm, 11.2 mm, 11.3 mm, 11.4 mm, 11.5 mm, 11.6 mm, 11.7 mm, 11.8 mm, 11.9 mm, 12 mm, 12.1 mm, 12.2 mm, 12.3 mm, 12.4 mm, 12.5 mm, 12.6 mm, 12.7 mm, 12.8 mm, 12.9 mm, 13 mm, 13.1 mm, 13.2 mm, 13.3 mm, 13.4 mm, 13.5 mm, 13.6 mm, 13.7 mm, 13.8 mm, 13.9 mm, 14 mm, 14.1 mm, 14.2 mm, 14.3 mm, 14.4 mm, 14.5 mm, 14.6 mm, 14.7 mm, 14.8 mm, 14.9 mm, 15 mm, 15.1 mm, 15.2 mm, 15.3 mm, 15.4 mm, 15.5 mm, 15.6 mm, 15.7 mm, 15.8 mm, 15.9 mm, 16 mm, 16.1 mm, 16.2 mm, 16.3 mm, 16.4 mm, 16.5 mm, 16.6 mm, 16.7 mm, 16.8 mm, 16.9 mm, 17 mm, 17.1 mm, 17.2 mm, 17.3 mm, 17.4 mm, 17.5 mm, 17.6 mm, 17.7 mm, 17.8 mm, 17.9 mm, 18 mm, 18.1 mm, 18.2 mm, 18.3 mm, 18.4 mm, 18.5 mm, 18.6 mm, 18.7 mm, 18.8 mm, 18.9 mm, 19 mm, 19.1 mm, 19.2 mm, 19.3 mm, 19.4 mm, 19.5 mm, 19.6 mm, 19.7 mm, 19.8 mm, 19.9 mm, or 20 mm. In other embodiments, the expanded diameter 216 can be less than approximately 0.01 mm or greater than approximately 20.00 mm. However, although the non-exclusive examples disclosed for the expanded diameter 216 are generally the same as the non-exclusive examples for the contracted diameter 214, it is appreciated that the expanded diameter 216 will be greater than the contracted diameter 214.

In certain embodiments, a ratio of the expanded diameter 216 to the contracted diameter 214 for the locator assembly 200 and/or the device body 212 herein can be greater than approximately 1:1 and less than or equal to approximately 20:1. In some such non-exclusive embodiments, the ratio of the expanded diameter 216 to the contracted diameter 214 for the locator assembly 200 and/or the device body 212 can be approximately 1.01:1, 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, 5:1, 5.1:1, 5.2:1, 5.3:1, 5.4:1, 5.5:1, 5.6:1, 5.7:1, 5.8:1, 5.9:1, 6:1, 6.1:1, 6.2:1, 6.3:1, 6.4:1, 6.5:1, 6.6:1, 6.7:1, 6.8:1, 6.9:1, 7:1, 7.1:1, 7.2:1, 7.3:1, 7.4:1, 7.5:1, 7.6:1, 7.7:1, 7.8:1, 7.9:1, 8:1, 8, 1:1, 8.2:1, 8.3:1, 8.4:1, 8.5:1, 8.6:1, 8.7:1, 8.8:1, 8.9:1, 9:1, 9.1:1, 9.2:1, 9.3:1, 9.4:1, 9.5:1, 9.6:1, 9.7:1, 9.8:1, 9.9:1, 10:1, 10.1:1, 10.2:1, 10.3:1, 10.4:1, 10.5:1, 10.6:1, 10.7:1, 10.8:1, 10.9:1, 11:1, 11.1:1, 11.2:1, 11.3:1, 11.4:1, 11.5:1, 11.6:1, 11.7:1, 11.8:1, 11.9:1, 12:1, 12.1:1, 12.2:1, 12.3:1, 12.4:1, 12.5:1, 12.6:1, 12.7:1, 12.8:1, 12.9:1, 13:1, 13.1:1, 13.2:1, 13.3:1, 13.4:1, 13.5:1, 13.6:1, 13.7:1, 13.8:1, 13.9:1, 14:1, 14.1:1, 14.2:1, 14.3:1, 14.4:1, 14.5:1, 14.6:1, 14.7:1, 14.8:1, 14.9:1, 15:1, 15.1:1, 15.2:1, 15.3:1, 15.4:1, 15.5:1, 15.6:1, 15.7:1, 15.8:1, 15.9:1, 16:1, 16.1:1, 16.2:1, 16.3:1, 16.4:1, 16.5:1, 16.6:1, 16.7:1, 16.8:1, 16.9:1, 17:1, 17.1:1, 17.2:1, 17.3:1, 17.4:1, 17.5:1, 17.6:1, 17.7:1, 17.8:1, 17.9:1, 18:1, 18.1:1, 18.2:1, 18.3:1, 18.4:1, 18.5:1, 18.6:1, 18.7:1, 18.8:1, 18.9:1, 19:1, 19.1:1, 19.2:1, 19.3:1, 19.4:1, 19.5:1, 19.6:1, 19.7:1, 19.8:1, 19.9:1, or 20:1. Alternatively, in other embodiments, the ratio of the expanded diameter 216 to the contracted diameter 214 for the locator assembly 200 and/or the device body 212 can be greater than approximately 20:1, or anywhere between 1:1 and 1.01:1.

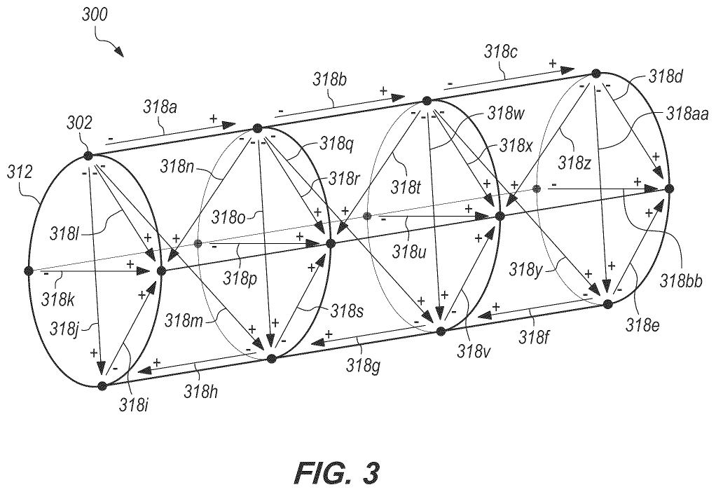

FIG. 3 is a simplified, partially transparent, perspective view of an embodiment of the locator assembly 300 illustrating bipolar relationships between electrodes 302 within the locator assembly 300. In the embodiment illustrated in FIG. 3, the locator assembly 300 can include the device body 312 and a plurality of bipoles 318a-318bb. The electrodes 302 can be bipolar electrodes having a negative polarity or positive polarity. In FIG. 3, the bipoles 318a-318bb are illustrated as vectors that indicate bipoles of electrical current running across the locator assembly 300 from the electrode 302 having a negative polarity to the electrode 302 having a positive polarity. The electrode 302 having a negative polarity is sometimes referred to herein as a cathode, and the electrode 302 having a positive polarity is sometimes referred to herein as an anode.

The bipoles 318a-318bb are formed between two electrical components (such as the anode and the cathode) with opposing polarities. In the bipoles 318a-318bb, the electrical current runs across the locator assembly 300 between the electrical components of opposing polarities. The electrodes 302 can be excited by applying a current or a voltage to produce the bipoles 318a-318bb between the anode and cathode. The current or the voltage can be applied to the electrodes 302 by the locator assembly 300 and/or the external device 105 (illustrated in FIG. 1).

The bipoles 318a-318bb can vary depending on the design requirements of the locator assembly 300 and/or the electrodes 302. In some embodiments, such as illustrated in FIG. 3, a network of bipoles 318a-318bb, including a plurality of anodes and cathodes, are arranged on the locator assembly 300. Multiple bipoles 318a-318bb or multiple bipole networks can be arranged in any suitable portion of the locator assembly 300. The bipoles 318a-318bb can be distributed about the longitudinal axis 100a (illustrated in FIG. 1A) in a pattern, either in the longitudinal and/or circumferential directions or about or along any other suitable axis.

In some embodiments, the bipoles 318a-318bb can be distributed in a somewhat circular, oval, cylindrical, or any other suitable pattern about the locator assembly 300. In one embodiment, the bipoles 318a-318bb can be evenly spaced apart from one another along the longitudinal axis 100a and/or about the circumference 100c (illustrated in FIG. 1A) of the locator assembly 300. In alternative embodiments, the bipoles 318a-318bb can be spaced apart from one another along the longitudinal axis 300a and/or about the circumference 100c of the locator assembly 300 in an uneven, semi-random, or random manner. While 28 bipoles 318a-318bb are displayed in the embodiment shown in FIG. 3, it is understood that more than 28 bipoles 318a-318bb or fewer than 28 bipoles 318a-318bb can be utilized by the locator assembly 300.

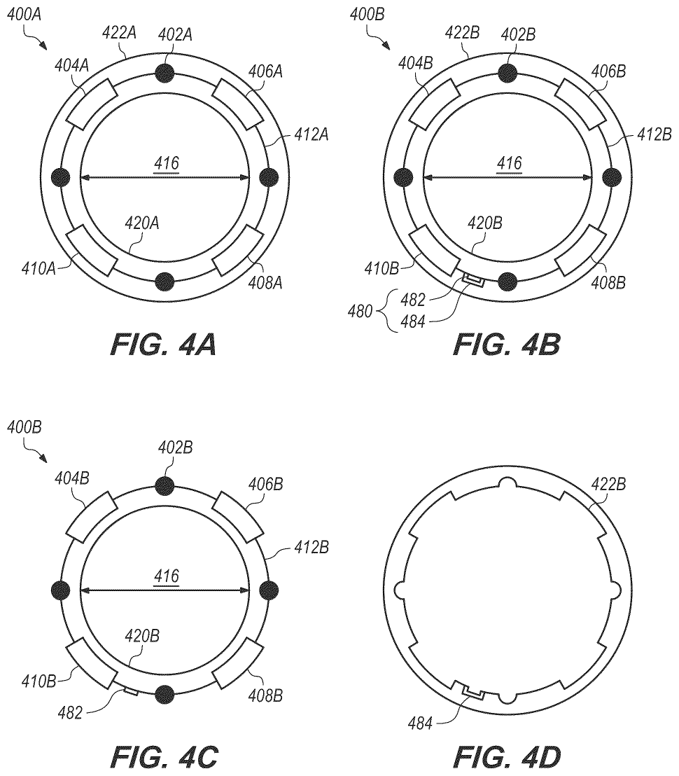

FIG. 4A is a simplified front elevation view of an embodiment of the locator assembly 400A. In particular, in the embodiment illustrated in FIG. 4A, the locator assembly 400A includes an inner layer 420A and an outer layer 422A. The inner layer 420A and the outer layer 422A can work in cooperation with one another to substantially enclose and/or protect the components of the locator assembly 400A, including the electrodes 402A, the communicator 404A, the controller 406A, the routing layer 408A, the power source 410A, and/or the device body 412A.

In other embodiments, the inner layer 420A can be coupled to the outer layer 422A to fully enclose the components of the locator assembly 400A, including the electrodes 402A, the communicator 404A, the controller 406A, the routing layer 408A, the power source 410A, and/or the device body 412A. In certain embodiments, only one layer (the inner layer 420A or the outer layer 422A) can fully enclose the components of the locator assembly 400A, including the electrodes 402A, the communicator 404A, the controller 406A, the routing layer 408A, the power source 410A, and/or the device body 412A.

The inner layer 420A and the outer layer 422A can cooperate with one another to improve the protection of the patient and the components of the locator assembly 400A upon deployment of the locator assembly 400A within the patient. The inner layer 420A can provide a substantially uniform surface to improve the protection of a deployment balloon 526 (illustrated in FIG. 5A) upon contact of the inner layer 420A with the deployment balloon 526. The outer layer 422A can reduce the likelihood of injury upon the contact of the outer layer 422A with one or more inner walls of portions of the heart 101 (illustrated FIG. 1B).

The inner layer 420A and/or the outer layer 422A can be in electrical communication with the electrodes 402A and the heart 101. The inner layer 420A and the outer layer 422A can be at least partially formed from electrically conductive materials. In other embodiments, the inner layer 420A and/or the outer layer 422A can be formed with holes or apertures that are configured to allow the electrodes 402A to come in direct contact with one or more inner walls of portions of the heart 101.

In some embodiments, the inner layer 420A and/or the outer layer 422A can release an eluting drug over a period of time to counteract the pro-thrombotic and inflammatory potential of the locator assembly 400A at its deployed location (for example, one deployed location is depicted in FIGS. 5A-5B). In other embodiments, one or more drug-eluting layers (not shown in FIG. 4A) can be coupled to each of the inner layer 420A and/or the outer layer 422 so that the inner layer 420A and/or the outer layer 422A are positioned between the one or more drug-eluting layers and the device body 412A. In certain embodiments, the inner layer 420A and/or the outer layer 422A can include multiple layers, including one or more drug-eluting layers. In various embodiments, other components of the locator assembly 400A (such as the device body 412A) can include an eluting drug and/or one or more drug-eluting layers.

Additionally, in the embodiment displayed in FIG. 4A, the locator assembly 400A is shown in the expanded state, where the locator assembly 400A has an expanded diameter 416. While the expanded state is shown in FIG. 4A, it is appreciated that the inner layer 420A and the outer layer 422A can be movable between the contracted state and the expanded state. The inner layer 420A can vary depending on the design requirements of the locator assembly 400A. In some embodiments, the inner layer 420A can be positioned differently than what is specifically illustrated in FIG. 4A.

The inner layer 420A can be formed from any suitable material. In certain embodiments, the inner layer 420A can be at least partially formed from a lubricious material and/or a continuous material. The inner layer 420A can be resilient, stretchable, and/or flexible. In some embodiments, the inner layer 420A can be at least partially formed from a metal, a plastic, a composite, a polymer, a coating, a biocompatible material, and/or a biodegradable material. Non-limiting, non-exclusive examples of suitable metals that can form the inner layer 420A include iron, magnesium, zinc, and their corresponding alloys. Non-limiting, non-exclusive examples of suitable polymers that can be used to form the inner layer 420A include polylactic acid, tyrosine polycarbonate, salicylic acid, poly-DL-lactide, and everolimus.

The inner layer 420A can include drugs to counteract the pro-thrombotic and inflammatory potential of the locator assembly 400A, such as immunosuppressive and antiproliferative drugs. Specific non-limiting, non-exclusive drugs usable within the inner layer 420A include sirolimus, paclitaxel, and everolimus. However, it is appreciated that any suitable, elutable drug can be utilized within the inner layer 420A.

The outer layer 422A can vary depending on the design requirements of the locator assembly 400A. In some embodiments, the outer layer 422A can be positioned differently than what is specifically illustrated in FIG. 4A. The outer layer 422A can be formed from any suitable material. In certain embodiments, the outer layer 422A can be at least partially formed from a lubricious material and/or a continuous material. The outer layer 422A can be resilient, stretchable, and/or flexible. In some embodiments, the outer layer 422A can be at least partially from a metal, a plastic, a composite, a polymer, a coating, a biocompatible material, and/or a biodegradable material. Non-limiting, non-exclusive examples of suitable metals that can form the outer layer 422A include iron, magnesium, zinc, and their corresponding alloys. Non-limiting, non-exclusive examples of suitable polymers that can be used to form the outer layer 422A include polylactic acid, tyrosine polycarbonate, salicylic acid, poly-DL-lactide, and everolimus.

The outer layer 422A can include drugs to counteract the pro-thrombotic and inflammatory potential of the locator assembly 400A, such as immunosuppressive and antiproliferative drugs. Specific non-limiting, non-exclusive drugs usable within the outer layer 422A include sirolimus, paclitaxel, and everolimus. However, it is appreciated that any suitable, elutable drug can be utilized within the outer layer 422A.

FIG. 4B is a simplified end view of yet another embodiment of the locator assembly 400B. In particular, in the embodiment illustrated in FIG. 4B, the locator assembly 400B again includes an inner layer 420B and an outer layer 422B. The inner layer 420B and the outer layer 420B can work in cooperation with one another to substantially enclose and/or protect the components of the locator assembly 400B, including the electrodes 402B, the communicator 404B, the controller 406B, the routing layer 408B, the power source 410B, and/or the device body 412B. The locator assembly 400B, the electrodes 402B, the communicator 404B, the controller 406B, the routing layer 408B, the power source 410B, the device body 412B, the inner layer 420B and/or the outer layer 420B can be substantially similar to the locator assembly 400A, the electrodes 402A, the communicator 404A, the controller 406A, the routing layer 408A, the power source 410A, the device body 412A, the inner layer 420A and/or the outer layer 420A described with respect to FIG. 4A and the other embodiments described herein. Accordingly, such components will not again be described in detail.

In the embodiment shown in FIG. 4B, the locator assembly 400B is movable between a locked state and an unlocked state. In some embodiments, the locator assembly 400B can include a locking assembly 480 that coordinates and/or enables the movement between the locked state and the unlocked state.

It is appreciated that the locator assembly 400B is shown in the locked state in FIG. 4B. In some embodiments, while the locator assembly 400B is in the unlocked state, the outer layer 422B can be separately deployable from the rest of the locator assembly 400B. Thus, in certain embodiments, the outer layer 422B can remain deployed at a deployment location (such as the location displayed in FIGS. 5A-5B, and 6D) while the rest of the locator assembly 400B is removed from the deployment location. A new locator assembly 400B can then be deployed and engaged within the outer layer 422B at the deployment location. However, it is appreciated that the outer layer 422B remains coupled to the locator assembly 400B, the device body 412B and/or the inner layer 420B, and is thus not separately deployable, when the locator assembly 400B is in the locked state.

The locking assembly 480 can facilitate the locking and/or separating of the outer layer 422B with the remainder of the locator assembly 400B and/or the device body 412B. For example, in certain embodiments, the locking assembly 480 enables locking and/or separation of the outer layer 422B from the remainder of the locator assembly 400B and/or the device body 412B by mechanical manipulation of the deployment catheter 524 (illustrated in FIG. 5A). The deployment catheter 524 can lock and unlock the locking assembly 480 so that the locator assembly 400B is separately positionable with respect to the outer layer 422B. In other embodiments, the patient, the clinician, and/or the external device 105 (illustrated in FIG. 1) can lock and/or unlock the locking assembly 480 without the mechanical manipulation of the deployment catheter 524. For example, in some embodiments, the external device 105 can wirelessly transmit (such as via the communicator 404B) a locking command and/or an unlocking command to the locking assembly 480 in order to lock or unlock the locking assembly 480.

The locking assembly 480 can vary depending on the design requirements of the locator assembly 400B and/or the outer layer 422B. It is understood that the locking assembly 480 can include additional components, systems, subsystems, and elements other than those specifically shown and/or described herein. Additionally, or alternatively, the locking assembly 480 can omit one or more of the components, systems, subsystems, and elements that are specifically shown and/or described herein. In some embodiments, the locking assembly 480 and the various components of the locking assembly 480 can be positioned in a different manner than what is specifically illustrated in FIG. 4B.

In many embodiments, as shown, the locking assembly 480 can include a first locking mechanism 482 and a second locking mechanism 484. The first locking mechanism 482 and the second locking mechanism 484 are configured to selectively lock and/or engage each other so that the outer layer 422B is secured to the locator assembly 400B and/or the device body 412B. The first locking mechanism 482 can be coupled to the device body 412B and/or any other suitable component of the locator assembly 400B. The second locking mechanism 484 can be coupled to the outer layer 422B and/or any other suitable component of the locator assembly 400B.

While the locking assembly 480 includes two locking mechanisms in FIG. 4B, it is appreciated that the locking assembly 480 can include any number of locking and/or engagement structures or elements that allow the locking and/or engagement of the outer layer 422B to the locator assembly 400B and/or the device body 412B. In some embodiments, as non-limiting, non-exclusive examples, the first locking mechanism 482 and the second locking mechanism 484 can include one or more of a male/female hookup assembly, a teeth/recess assembly, a tongue/groove assembly, a latch, an anchor, a coupling, an interlocking shoulder, a bolt, a cable, a clamp, a connector, a hook, a loop, a flange protrusion, a joint, a seam, a channel, a guide, a linkage, a track, and/or a tray. The first locking mechanism 482 and the second locking mechanism 484 have been simplified as shown in FIG. 4B for ease of understanding.

FIG. 4C is a simplified end view of a portion of the embodiment of the locator assembly 400B shown in FIG. 4B. More particularly, as shown in FIG. 4C, the locator assembly 400B including the electrodes 402B, the communicator 404B, the controller 406B, the routing layer 408B, the power source 410B, the device body 412B, the inner layer 420B, and/or the first locking mechanism 482 can be selectively unlocked and/or detached from the outer layer 422B and/or the second locking mechanism 484.

FIG. 4D is a simplified end view of another portion of the embodiment of the locator assembly 400B shown in FIG. 4B. More particularly, FIG. 4D is a simplified end view of the outer layer 422B that can be included as part of the locator assembly 400B or can be configured to selectively engage the locator assembly 400B. As shown in FIG. 4D, the outer layer 422B and/or the second locking mechanism 484 can be selectively unlocked and/or detached from the electrodes 402B, the communicator 404B, the controller 406B, the routing layer 408B, the power source 410B, the device body 412B, the inner layer 420B, and/or the first locking mechanism 482.

FIG. 5A is a simplified, partially transparent view of an embodiment of the locator assembly 500 and embodiments of a deployment catheter 524 that includes a sheath 523, a guidewire 525, and an inflatable balloon 526 (also referred to herein as a “balloon”). As illustrated in FIG. 5A, the locator assembly 500 is positioned within the coronary sinus 527 of the heart 101 (illustrated in FIG. 1) near the vena cordis media 528. Alternatively, the locator assembly 500 can be positioned within another suitable portion of the heart, such as within the vein of Marshall in one non-exclusive alternative implementation.

As utilized herein, the locator assembly 500 and the deployment catheter 524 can be referred to collectively as a locator system 521.

The deployment catheter 524 deploys the locator assembly 500 in a portion of the heart 101. More particularly, the deployment catheter 524 initially deploys the locator assembly 500 in a deployment direction 529A (illustrated with an arrow) so that the locator assembly 500 is positioned at a desired location or target site within the heart 101, such as within the coronary sinus 527 and near the vena cordis media 528. Although not specifically shown in FIG. 5A, the locator assembly 500 will typically be positioned substantially fully within the sheath 523 of the deployment catheter 524 during the initial deployment of the locator assembly 500.

In various implementations, the deployment catheter 524 can deploy the locator assembly 500 in the same or similar manner as the deployment catheter 524 would deploy an expandable stent. As noted above, in some embodiments, the locator assembly 500 can incorporate and/or include such an expandable stent within its overall structure. The deployment catheter 524 can advance the locator assembly 500 to a target site within the coronary sinus 527. In some embodiments (such as the embodiment shown in FIG. 5A), the target site can be near a junction between the coronary sinus 527 and the vena cordis media 528. The target site illustrated in FIG. 5A is merely demonstrative, and it is appreciated that the locator assembly 500 can be deployed in any suitable position within the patient. The deployment catheter 524 can deploy the locator assembly 500 while the device is in the contracted state, the expanded state, or between states (in FIG. 5A, the locator assembly 500 is shown in the contracted state). It is appreciated, however, that the initial deployment will often occur with the locator assembly 500 in the contracted state, as that would make for the easiest navigation through the structures of the heart 101 or other locations within the body of the patient.

The deployment catheter 524 can vary depending on the design requirements of the locator assembly 500. In various embodiments, as shown, the deployment catheter 524 includes the sheath 523 that provides structural protection for the locator assembly 500 as the locator assembly 500 is initially deployed within the heart 101. It is understood that the deployment catheter 524 can include additional components, systems, subsystems, and elements other than those specifically shown and/or described herein. Additionally, or alternatively, the deployment catheter 524 can omit one or more of the components, systems, subsystems, and elements that are specifically shown and/or described herein. In particular, the deployment catheter 524 in FIG. 5A has been simplified, and some elements of the deployment catheter 524 have been omitted for ease of understanding. In some embodiments, the deployment catheter 524 can be positioned differently than what is specifically illustrated in FIG. 5A.

In some embodiments, the deployment catheter 524 can be a percutaneous transcatheter or any suitable catheter. As shown, the deployment catheter 524 can further include the guidewire 525 and the inflatable balloon 526. The deployment catheter 524 and/or the sheath 523 can be configured to move over the guidewire 525. During initial deployment, the balloon 526 and the locator assembly 500 can be positioned substantially fully within the sheath 523 until the target site is reached. Subsequently, as described in greater detail herein below, the balloon 526 and the locator assembly 500 can then be left in position at the target site while the sheath 523 is being retracted and/or removed from within the body of the patient.

The guidewire 525 can advance components (such as the locator assembly 500 and/or the balloon 526) through an opening of the deployment catheter 524 and/or the sheath 523. The guidewire 525 can be advanced simultaneously with the deployment catheter 524 and/or the sheath 523 within the body of the patient. The guidewire 525 can vary depending on the design requirements of the locator assembly 500 and/or the deployment catheter 524. In some embodiments, the guidewire 525 can be positioned differently than what is specifically illustrated in FIG. 5A.

The balloon 526 can be coupled to the deployment catheter 524 and/or the guidewire 525. The balloon 526 can be inflatable to move the locator assembly 500 between the contracted state and the expanded state. The balloon 526 can be deflated and removed from the interior of the locator assembly 500 after the locator assembly 500 has been moved to the contracted state from the expanded state. The balloon 526 can also be deflated and removed from the interior of the locator assembly 500 when the locator assembly 500 is in between the contracted state and the expanded state.

The balloon 526 can vary depending on the design requirements of the locator assembly 500, the deployment catheter 524, and/or the guidewire 525. In some embodiments, the balloon 526 can be positioned differently than what is specifically illustrated in FIG. 5A. The balloon 526 illustrated in FIG. 5A has been simplified for ease of understanding.

FIG. 5B is a simplified, partially transparent view of an embodiment of the locator assembly 500 and an embodiment of the deployment catheter 524 including the sheath 523, the guidewire 525, and the balloon 526. In the embodiment shown in FIG. 5B, the locator assembly 500 is positioned within the coronary sinus 527 of the heart 101 (illustrated in FIG. 1), and the locator assembly 500 is now shown in the expanded state. More particularly, as illustrated, with the locator assembly 500 and the balloon 526 positioned at the target site, the sheath 523 has been partially retracted, in a sheath retraction direction 529B (illustrated with an arrow), such that the locator assembly 500 and the balloon 526 are no longer positioned within the sheath 523. With the locator assembly 500 and the balloon 526 no longer positioned within the sheath 523, the balloon 526 can then be inflated so as to move the locator assembly 500 from the contracted state (such as shown in FIG. 5A) to the expanded state (such as shown in FIG. 5B). Stated in another manner, in this embodiment, the locator assembly 500 and the balloon 526 can be configured such that inflation of the balloon 526 can move the locator assembly 500 from the contracted state to the expanded state. Alternatively, the locator assembly 500 can be moved from the contracted state to the expanded state in another suitable manner. For example, in one non-exclusive alternative embodiment, such as shown and described herein below in relation to FIGS. 6A-6C, the locator assembly 500 can be spontaneously self-expanding, such that the locator assembly 500 will spontaneously move from the contracted state to the expanded state as the sheath 523 is retracted, and the locator assembly 500 is no longer positioned within the sheath 523. It is appreciated that in such alternative embodiment, the deployment catheter 524 would typically be designed without the balloon 526, as the balloon 526 is no longer required for moving the locator assembly 500 from the contracted state to the expanded state.

In certain embodiments, the guidewire 525 can be utilized to extract the balloon 526 from the interior of the locator assembly 500. In some embodiments, the balloon 526 can first be deflated so that it can more readily retract within the interior of the deployment catheter 524 and/or the sheath 523.

FIG. 6A is a simplified, partially transparent view of another embodiment of the locator assembly 600 including a device body 612, and another embodiment of the deployment catheter 624 that includes a sheath 623 and a guidewire 625. In FIG. 6A, the locator assembly 600 is being shown in an initial deployment position. As illustrated in FIG. 6A, the locator assembly 600 is positioned within the coronary sinus 627 of the heart 101 (illustrated in FIG. 1) near the vena cordis media 628.

As above, the locator assembly 600 and the deployment catheter 624 can again be referred to collectively as a locator system 621.

In this embodiment, the sheath 623 and the guidewire 625 of the deployment catheter 624 are substantially similar to the previous embodiments. However, the locator assembly 600 can have a somewhat different design than the previous embodiments. In particular, in this embodiment, the locator assembly 600 and/or the device body 612 can include and/or incorporate a spontaneous, passive, self-expanding design that was not present in the previous embodiments. For this reason, the deployment catheter 624 as shown in FIG. 6A does not include a balloon, as a balloon is no longer required to move the locator assembly 600 and/or the device body 612 from a contracted state to an expanded state.

As with the previous embodiments, the deployment catheter 624 deploys the locator assembly 600 in a portion of the heart 101. More particularly, the deployment catheter 624 initially deploys the locator assembly 600 in a deployment direction 629A (illustrated with an arrow) so that the locator assembly 600 is positioned at a desired location or target site within the heart 101, such as within the coronary sinus 627 and/or near the vena cordis media 628. Alternatively, the desired location or target site can be at a different location within the body of the patient, such as in the vein of Marshall in one non-exclusive implementation.

As shown in FIG. 6A, with the locator assembly 600 in the initial deployment position, the locator assembly 600 will typically be positioned substantially fully within the sheath 623 of the deployment catheter 624 during such initial deployment of the locator assembly 600. When so positioned fully within the sheath 623, the locator assembly 600 and/or the device body 612 will be in the contracted state, as the structure of the sheath 623 will compress the locator assembly 600 into a more compact configuration, and inhibit the locator assembly 600 and/or the device body 612 from moving to the expanded state.

In various implementations, the deployment catheter 624 can deploy the locator assembly 600 in the same or similar manner as the deployment catheter 624 would deploy an actively expandable stent, such as a stent that is moved to an expanded state using another structures, such as a balloon, for example. As noted above, in some embodiments, the locator assembly 600 and/or the device body 612 can incorporate and/or include such an expandable stent within its overall structure. The deployment catheter 624 can advance the locator assembly 600 to a target site within the coronary sinus 627. In some embodiments (such as the embodiment shown in FIG. 6A), the target site can be near a junction between the coronary sinus 627 and the vena cordis media 628. The target site illustrated in FIG. 6A is merely demonstrative, and it is appreciated that the locator assembly 600 can be deployed in any suitable position within the patient. The deployment catheter 624 can deploy the locator assembly 600 while the device is in the contracted state, the expanded state, or between states (in FIG. 6A, the locator assembly 600 is shown in the contracted state). It is appreciated, however, that the initial deployment will most often occur with the locator assembly 600 in the contracted state, as that would provide for the easiest navigation through the structures of the heart 101 or other locations within the body of the patient.

The deployment catheter 624 can vary depending on the design requirements of the locator assembly 600. As shown, the deployment catheter 624 can be somewhat similar to the deployment catheter 524 illustrated and described herein above in relation to FIGS. 5A-5B. For example, in various embodiments, the deployment catheter 624 again includes the sheath 623 that provides structural protection for the locator assembly 600 as the locator assembly 600 is initially deployed within the heart 101. It is understood that the deployment catheter 624 can include additional components, systems, subsystems, and elements other than those specifically shown and/or described herein. Additionally, or alternatively, the deployment catheter 624 can omit one or more of the components, systems, subsystems, and elements that are specifically shown and/or described herein. In particular, the deployment catheter 624 in FIG. 6A has been simplified, and some elements of the deployment catheter 624 have been omitted for clarity and/or ease of understanding. In some embodiments, the deployment catheter 624 can be positioned differently than what is specifically illustrated in FIG. 6A.

In some embodiments, the deployment catheter 624 can be a percutaneous transcatheter or any suitable catheter. As shown, the deployment catheter 624 can further include the guidewire 625. The deployment catheter 624 and/or the sheath 623 can be configured to move over the guidewire 625. During initial deployment, the locator assembly 600 can be positioned substantially fully within the sheath 623 until the target site is reached. Subsequently, as described in greater detail herein below, the locator assembly 600 can then remain in position at the target site while the sheath 623 is being retracted and/or removed from within the body of the patient.

The guidewire 625 can advance components (such as the locator assembly 600) through an opening of the deployment catheter 624 and/or the sheath 623. The guidewire 625 can be advanced simultaneously with the deployment catheter 624 and/or the sheath 623 within the body of the patient. The guidewire 625 can vary depending on the design requirements of the locator assembly 600 and/or the deployment catheter 624. In some embodiments, the guidewire 625 can be positioned differently than what is specifically illustrated in FIG. 6A.

However, as noted above, in this embodiment, the locator assembly 600 has a different design than in the previous embodiments. In particular, the locator assembly 600 shown in FIG. 6A has a spontaneous and/or passive, self-expanding design such that the locator assembly 600 passively expands from the contracted state to the expanded state when not otherwise constrained, such as when the locator assembly 600 is maintained in the contracted state due to its position within the sheath 623 of the deployment catheter 624. More specifically, as shown in FIG. 6A, the locator assembly 600 is advanced to a desired location or target site, such as into the coronary sinus 627 or other location within the body of the patient, via the delivering, pre-formed sheath 623, with the locator assembly 600 positioned therewithin. Subsequently, as described in greater detail herein below, upon retraction and/or retrieval of the sheath 623, the locator assembly 600, and the stent structure of the device body 612 included therein, is deployed spontaneously and remains at the target site according to its memory properties. The locator assembly 600 then stays in position as a chronic device at the target site. Thus, as compared to the previous embodiments for chronic assembly delivery that used an active balloon-mediated method, this embodiment provides the possibility for a passive chronic assembly delivery option.

FIG. 6B is a simplified, partially transparent view of the locator assembly 600 and the deployment catheter 624 illustrated in FIG. 6A, the locator assembly 600 now being shown in a partially deployed position. As shown in FIG. 6B, the locator assembly 600 is again shown positioned within the coronary sinus 627 of the heart 101 (illustrated in FIG. 1) and near the vena cordis media 628.

As illustrated in FIG. 6B, the sheath 623 of the deployment catheter 624 has been partially retracted in a sheath retraction direction 629B (illustrated with an arrow) so that approximately half of the locator assembly 600 is still positioned within the sheath 623, while the other half of the locator assembly 600 is now positioned outside of the sheath 623. This is considered as the partially deployed position. It is appreciated, however, that the partially deployed position can include any time along a continuum when some portion of the locator assembly 600 is now positioned outside of the sheath 623, while the other portion, or remainder, of the locator assembly 600 is still positioned within the sheath 623.

As shown, as the structure of the locator assembly 600 and/or the device body 612 moves out from within the constraints of and/or is removed from the sheath 623, the locator assembly 600 and/or the device body 612 spontaneously moves from the contracted state toward the expanded state. In particular, FIG. 6B illustrates that the portion of the locator assembly 600 and/or the device body 612 that is no longer positioned within and/or has been removed from the sheath 623 has moved from the contracted state toward the expanded state, while the other portion of the locator assembly 600 and/or the device body 612 that is still positioned within the sheath 623 is still maintained in the contracted state. With this design, the locator assembly 600 and/or the device body 612 can be said to include, can function as and/or can be referred to as a “self-expanding stent” or a “self-expandable stent”.

FIG. 6C is a simplified, partially transparent view of the locator assembly 600 and the deployment catheter 624 illustrated in FIG. 6A, the locator assembly 600 now being shown in a fully deployed position. As shown in FIG. 6C, the locator assembly 600 is again shown positioned within the coronary sinus 627 of the heart 101 (illustrated in FIG. 1) and near the vena cordis media 628. In particular, the locator assembly 600 and/or the device body 612 is now positioned at the desired location or target site as a chronic stent-like device.

As illustrated in FIG. 6C, the sheath 623 of the deployment catheter 624 has been fully retracted relative to the locator assembly 600 in the sheath retraction direction 629C (illustrated with an arrow) so the entirety of the locator assembly 600 is now positioned outside of the sheath 623. This is considered as the fully deployed position.

With the locator assembly 600 now positioned fully outside the sheath 623, the spontaneous self-expanding design of the locator assembly 600 has resulted in the locator assembly 600 and/or the device body 612 moving fully from the contracted state to the expanded state. Thus, in comparison to the embodiment of FIGS. 5A-5B which focuses on the possibility for chronic stent delivery using an active balloon-mediated method, the embodiment of FIGS. 6A-6C focuses on the possibility for chronic stent delivery using a passive, self-expanding method.

FIG. 6D is a simplified illustration of the heart 601, including the right atrium 601a and the left atrium 601b, and an embodiment of the locator assembly 600D for determining the location of the arrhythmogenic foci 632 in or near the heart 601. It is appreciated that the locator assembly 600D shown in FIG. 6D can be in the form of the embodiment of the locator assembly 600 illustrated and described in relation to FIGS. 6A-6C. Alternatively, it is further appreciated that the locator assembly 600D shown in FIG. 6D can be in the form of the embodiment of the locator assembly 500 illustrated and described in relation to FIGS. 5A-5B.

In FIG. 6D, the locator assembly 600D is positioned within a portion of the heart 601. For ease in understanding, FIG. 6D displays exemplar locations of a sinus rhythm foci 630, the arrhythmogenic foci 632, and a predicted foci 634 in the heart 601.

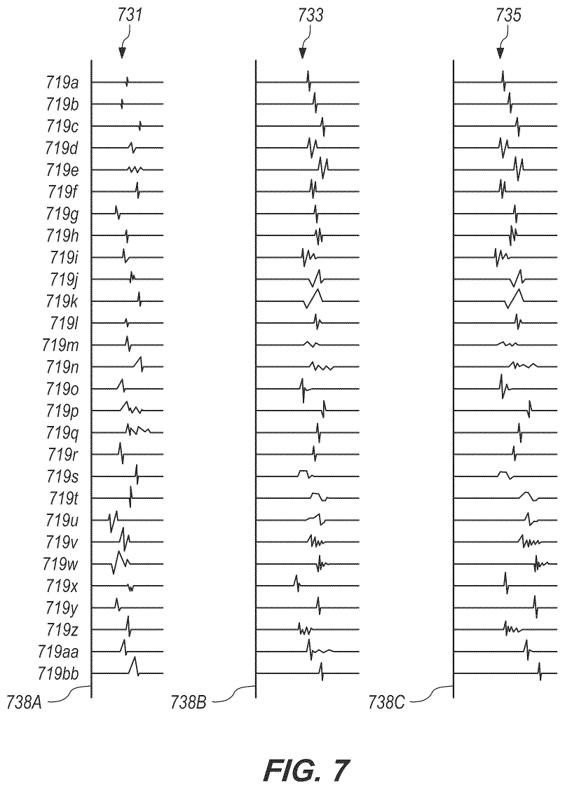

The sinus rhythm foci 630 is the focal point of a normal sinus rhythm of the patient. In particular, in some embodiments, the sinus rhythm foci 630 represents the origin of the electrical activation sequences of the normal sinus rhythm, such as from the sino-atrial node. One example of electrical activation sequence signal arrays recorded by the locator assembly 600 at the sinus rhythm foci 630 is illustrated in FIG. 7 in the left column.

The arrhythmogenic foci 632 illustrated in FIG. 6D is representative of one actual location of the focal point of an arrhythmia of the patient. It is appreciated that the arrhythmogenic foci 632 shown in FIG. 6D is merely demonstrative and/or representative, and can be located anywhere in and/or near the heart 601.

The predicted foci 634 in FIG. 6D represents the location of an artificial stimulation to determine and/or confirm whether the predicted foci 634 is the same or different than the actual arrhythmogenic foci 632. The predicted foci 634 and the arrhythmogenic foci 632 can be located at the same location (referred to herein as a “matched state”) or different locations (referred to herein as an “unmatched state”), as described in greater detail herein.

The artificial stimulation can be generated using any suitable device known in the art, including ablation catheters, electrical stimulation and/or pacemakers, as non-exclusive examples. The artificial stimulation device can stimulate any suitable number of predicted foci 634 during one operation and/or insertion of the artificial stimulation device into the patient. In other words, the artificial stimulation device can test various predicted foci 634 locations in rapid succession.

FIG. 7 is a simplified diagram displaying electrical signal array data collected by the locator assembly 100 (illustrated in FIG. 1, for example). As shown in FIG. 7, the signal array data collected by the bipoles 318a-318bb (illustrated in FIG. 3) is illustrated in descending rows as electrical signals 719a-719bb. For example, FIG. 7 illustrates a sinus signal array 731 that is collected by the locator assembly 100 from the sinus rhythm foci 630 (illustrated in FIG. 6), a first signal array 733 that is collected by the locator assembly 100 from the arrhythmogenic foci 632 (illustrated in FIG. 6), and a second signal array 735 that is collected from the locator assembly 100 from the predicted foci 634 (illustrated in FIG. 6).

FIG. 7 illustrates the sinus signal array 731, the first signal array 733, and the second signal array 735 that can be displayed on a graphical user interface (GUI) of the external device 105 (illustrated in FIG. 1). It is understood that the actual display of the sinus signal array 731, the first signal array 733, and/or the second signal array 735 can appear differently than those shown in FIG. 7, and that the sinus signal array 731, the first signal array 733 and the second signal array 735 illustrated in FIG. 7 are provided as representative of one type of display for ease in understanding, and are not intended to be limiting in any manner. Any other suitable visual and/or auditory displays are contemplated and are intended to be included as alternative embodiments. Still alternatively, a haptic response can be incorporated into the display of the sinus signal array 731, the first signal array 733, and the second signal array 735.

The sinus signal array 731 illustrates the electrical activation sequence recorded by the locator assembly 100 implanted in the coronary sinus 127 (illustrated in FIG. 1B) during the patient's normal sinus rhythm. In particular, the sinus signal array 731 is recorded by each of the bipoles 318a-318bb to generate corresponding electrical signals 719a-719bb in descending rows. For example, the bipole 318a receives the electrical signal 719a from the sinus rhythm foci 630 during the patient's normal sinus rhythm, which is then displayed in the first row of the sinus signal array 731. Each additional bipole 318b-318bb likewise receives the corresponding electrical signal 719b-719bb, which are likewise displayed in subsequent rows in the sinus signal array 731.

The sinus signal array 731 can be used for a comparative assessment of the different sequences between the two alternative sources of a cardiac impulse origin. In particular, the sinus signal array 731 can represent the patient's electrical activation sequence of the sinus rhythm. The sinus signal array 731 can be used in comparison with the first signal array 733 and/or the second signal array 735.

In the embodiment illustrated in FIG. 7, the sinus signal array 731 includes an event initiation 738A represented as a vertical straight line in the sinus signal array 731. The event initiation 738A represents the onset of an actual event, such as an electrophysiological event including an electrical signal originating from the sinus rhythm foci (or sino-atrial node), for example. It is understood that the event initiation 738A represents a time (such as at T₀), after which electrical signals 719a-719bb occur.

The first signal array 733 illustrates the electrical activation sequence located at the arrhythmogenic foci 632 and recorded by the locator assembly 100 implanted in the coronary sinus 127 during a clinical episode of atrial fibrillation of the patient. In particular, the first signal array 733 is recorded by each of the bipoles 318a-318bb to generate corresponding electrical signals 719a-719bb in descending rows. For example, the bipole 318a records the electrical activation sequence during a clinical episode of atrial fibrillation of the patient, and the corresponding electrical signal 719a is displayed in the first row of the first signal array 733. Each additional bipole 318b-318bb likewise receives the corresponding electrical signal 719b-719bb, which are likewise displayed in subsequent rows in the first signal array 733. The first signal array 733 can be used in comparison with the second signal array 735, as provided in greater detail herein.

It is appreciated that the arrhythmogenic foci 632, as located through use of the locator assembly 100 and incorporated within the first signal array 733, can be recorded at any suitable time(s) during the time interval between implantation of the locator assembly 100 within the heart 101 (illustrated in FIG. 1) of the patient and any subsequent ablation procedure. For example, in some implementations, the arrhythmogenic foci 632 can be recorded during a spontaneously initiated episode of clinical atrial fibrillation during such time interval using the technology exemplified in FIGS. 1-4, and/or can be recorded from occasional occurrences in the setting of the ablation procedure using the technology exemplified in FIGS. 5 and 6.

In the embodiment illustrated in FIG. 7, the first signal array 733 includes an event initiation 738B represented as a vertical straight line in the first signal array 733. The event initiation 738B represents the initiation of an actual event, such as an electrophysiological event including an electrical signal originating from the arrhythmogenic foci 632, for example. It is understood that the event initiation 738B represents a time (such as at T₀), after which electrical signals 719a-719bb occur.

The second signal array 735 illustrates the electrical activation sequence taken at the predicted foci 634 and recorded by the locator assembly 100 implanted in the coronary sinus 127 during artificial stimulation of the patient at the predicted foci 634. In particular, the second signal array 735 is recorded by each of the bipoles 318a-318bb to corresponding electrical signals 719a-719bb in descending rows. For example, the bipole 318a records the electrical activation sequence during artificial stimulation of the patient at the predicted foci 634, and the corresponding electrical signal 719a is displayed on the first row in the second signal array 735. Each additional bipole 318b-318bb likewise receives the corresponding electrical signal 719b-719bb, which are likewise displayed in subsequent rows in the second signal array 735. The second signal array 735 can be used in comparison with the first signal array 733, as provided in greater detail herein.

In the embodiment illustrated in FIG. 7, the second signal array 735 includes an event initiation 738C represented as a vertical straight line in the second signal array 735. The event initiation 738C represents the initiation of an actual event, such as an artificial stimulus that generates an electrical signal originating from the predicted foci 634, for example. It is understood that the event initiation 738C represents a time (such as at T₀), after which electrical signals 719a-719bb occur.

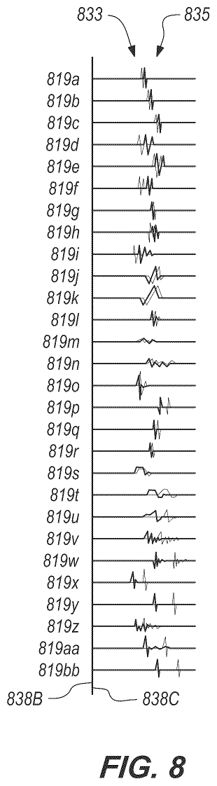

FIG. 8 is a simplified diagram illustrating a superimposition of the first signal array 833 and the second signal array 835 over one another generated during one embodiment of the method for locating the arrhythmogenic foci 632 (illustrated in FIG. 6D). In FIG. 8, the superimposition is shown in an unmatched state. As shown in the embodiment displayed in FIG. 8, the electrical signals 819a-819bb are recorded by the bipoles 318a-318bb (illustrated in FIG. 3) in descending rows.

In the embodiment illustrated in FIG. 8, the first signal array 833 includes the event initiation 838B represented as a vertical straight line in the first signal array 833, and the second signal array 835 includes the event initiation 838C represented as a vertical straight line in the second signal array 835. In this embodiment, the event initiations 838B, 838C are aligned so a direct comparison between the first signal array 833 and the second signal array 835 can be achieved. Based on the superimposition displayed in FIG. 8, the clinician and/or the patient can determine and/or confirm that the arrhythmogenic foci 632 (as recorded during a spontaneously initiated episode of clinical atrial fibrillation occurring during the time interval between stent implantation and ablation using the technology exemplified in FIGS. 1-4, or as occurring occasionally in the setting of the ablation procedure using the technology exemplified in FIGS. 5 and 6) and the predicted foci 634 (illustrated in FIG. 6D) are not in the same location as one another. In this embodiment, the signal arrays 833, 835 are not substantially similar or identical.

A negative sensory response can be incorporated into the locator assembly 100 (illustrated in FIG. 1), the deployment catheter 524 (illustrated in FIG. 5A), and/or a related system in order to assist the clinician and/or the patient in determining that the arrhythmogenic foci 632 and the predicted foci 634 are not in the same location as one another (that is, are in the unmatched state). For example, in some embodiments, a negative haptic response can be incorporated into the display of the signal arrays 833, 835, or into a handle (not shown) of the deployment catheter 524. The negative haptic response can be a vibration or similar stimulation of touch and/or motion. The negative haptic response can be included on any suitable portion of the deployment catheter 524 or any suitable system and/or device. In other embodiments, a negative audio response can include a beep or any suitable audio feedback that is triggered when the superimposition is in the unmatched state. In certain embodiments, a negative visual response can include a red visual indicator and/or any suitable visual indication when the superimposition is in the unmatched state.

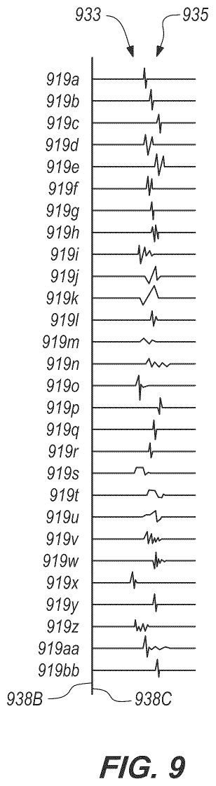

FIG. 9 is a simplified diagram illustrating a superimposition of the first signal array 933 and the second signal array 935 over one another generated during an embodiment of the method for determining the location of the arrhythmogenic foci 632 (illustrated in FIG. 6D), the superimposition being shown in a matched state. As shown in the embodiment displayed in FIG. 9, the electrical signals 919a-919bb are recorded by the bipoles 318a-318bb (illustrated in FIG. 3) in descending rows.

In the embodiment illustrated in FIG. 9, the first signal array 933 includes the event initiation 938B represented as a vertical straight line in the first signal array 933, and the second signal array 935 includes the event initiation 938C represented as a vertical straight line in the first signal array 935. In this embodiment, the event initiations 938B, 938C are aligned so a direct comparison between the first signal array 933 and the second signal array 935 can be achieved. Based on the superimposition displayed in FIG. 9, the clinician and/or the patient can determine and/or confirm that the arrhythmogenic foci 632 and the predicted foci 634 (illustrated in FIG. 6D) are in the same location as one another because the signal arrays 833, 835, are substantially similar or identical.

A positive sensory response can be incorporated into the locator assembly 100 (illustrated in FIG. 1), the deployment catheter 524 (illustrated in FIG. 5A), and/or a related system, in order to assist the clinician and/or the patient in determining that the arrhythmogenic foci 632 and the predicted foci 634 are in the same location as one another (that is, are in the matched state). For example, in some embodiments, a positive haptic response can be incorporated into the display of the signal arrays 833, 835, or into a handle (not shown) of the deployment catheter 524. The positive haptic response can be a vibration or similar stimulation of touch and/or motion. The positive haptic response can be included on any suitable portion of the deployment catheter 524 or any suitable system and/or device. In other embodiments, a positive audio response can include a beep or any suitable audio feedback that is triggered when the superimposition is in the matched state. In certain embodiments, a positive visual response can include a green visual indicator and/or any suitable visual indication when the superimposition is in the matched state.

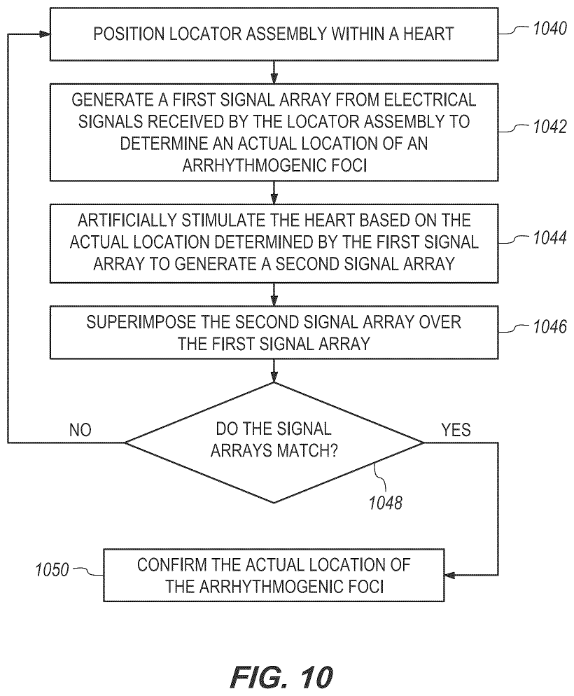

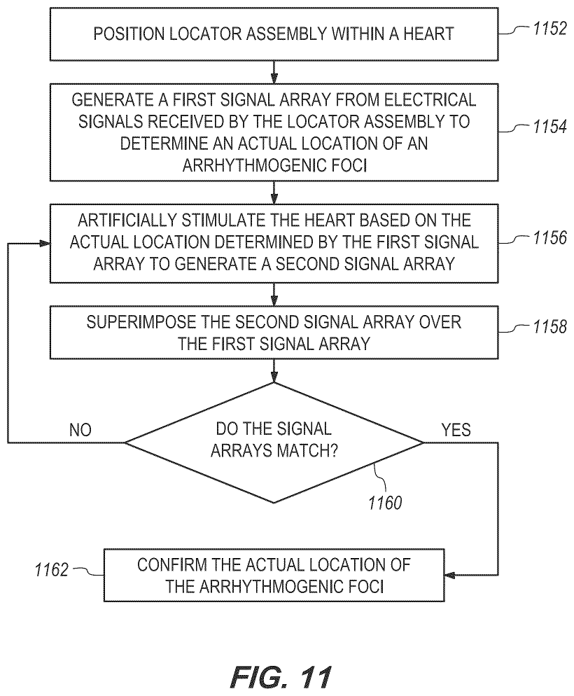

FIG. 10 is a flow chart outlining one embodiment of a method for determining the location of arrhythmogenic foci in the heart. It is understood that the method pursuant to the disclosure herein can include greater or fewer steps than those shown and described relative to FIG. 10. The method can omit one or more steps illustrated in FIG. 10. The method can add additional steps not shown and described in FIG. 10, and still fall within the purview of the present invention. Further, the sequence of the steps can be varied from those shown and described relative to FIG. 10. The sequence of steps illustrated in FIG. 10 is not intended to limit the sequencing of steps in any manner.

In the embodiment illustrated in FIG. 10, at step 1040, a locator assembly is positioned within the heart. In certain embodiments, the locator assembly can include and/or incorporate an expandable stent that is configured to be inserted into the heart. The locator assembly can include a plurality of electrodes that receive electrical signals from the heart. The locator assembly can be positioned within the heart using any suitable method known within the art, including those described herein. In some implementations, the locator assembly can be positioned within a coronary sinus of the patient. Alternatively, in other implementations, the locator assembly can be positioned in other suitable areas within the heart. Still alternatively, other designs of locator assemblies can be utilized by the methods described herein.

At step 1042, a first signal array is generated from the electrical signals recorded by the locator assembly to determine the actual location of the arrhythmogenic foci. The locator assembly can use a plurality of electrodes arranged in bipolar relationships to receive the electrical signals. The electrical signals recorded by the plurality of electrodes can include atrial electrical activation signals. As used herein, the arrhythmogenic foci can also include any focal location within a human body associated with the development or perpetuation of atrial fibrillation.

At step 1044, the heart is artificially stimulated based on the actual location of the arrhythmogenic foci determined by the first signal array to generate a second signal array. The heart can be artificially stimulated by any suitable device known in the art. The second signal array can include the electrical activation sequence taken at the predicted foci and recorded by the locator assembly during a clinical episode of atrial fibrillation of the patient.

At step 1046, the second signal array is superimposed over the first signal array. The superimposition of the signal array data can be completed in the same and/or a similar manner as the embodiments illustrated in FIGS. 8-9. In some embodiments, the step of superimposing can be displayed on a graphical user interface (GUI) on an external device.

At step 1048, the superimposed signal arrays are compared. If the signal arrays match, the method proceeds to step 1050. If the signal arrays are not matched, the method restarts at step 1040. Stated in another manner, in the method of FIG. 10, if the signal arrays are not matched, then the locator assembly is positioned in a different manner and/or at a different location within the heart, and then steps 1042-1048 are repeated.

At step 1050, the actual location of arrhythmogenic foci is confirmed, and the method for determining the location of arrhythmogenic foci in or near the heart is completed.

FIG. 11 is a flow chart outlining another embodiment of a method for determining the location of arrhythmogenic foci in the heart. It is understood that the method pursuant to the disclosure herein can include greater or fewer steps than those shown and described relative to FIG. 11. The method can omit one or more steps illustrated in FIG. 11. The method can add additional steps not shown and described in FIG. 11, and still fall within the purview of the present invention. Further, the sequence of the steps can be varied from those shown and described relative to FIG. 11. The sequence of steps illustrated in FIG. 11 is not intended to limit the sequencing of steps in any manner.

In the embodiment illustrated in FIG. 11, at step 1152, a locator assembly is positioned within the heart. It is appreciated, as noted above, that the locator assembly can be positioned at any suitable location within the heart, such as in the coronary sinus in one non-exclusive implementation. The locator assembly can include a plurality of electrodes that receive electrical signals from the heart. However, other designs of locator assemblies can be used with the methods described herein.

At step 1154, a first signal array is generated from the electrical signals received by the locator assembly to determine an actual location of the arrhythmogenic foci.

At step 1156, the heart is artificially stimulated based on the actual location of the arrhythmogenic foci determined by the first signal array to generate a second signal array. The heart can be artificially stimulated by any suitable device known in the art.

At step 1158, the second signal array is superimposed over the first signal array. The superimposition of the signal data can be the same and/or similar to the embodiments illustrated in FIGS. 8-9. In some embodiments, the step of superimposing can be displayed on a graphical user interface (GUI) on an external device.

At step 1160, the superimposed signal arrays are compared. If the signal arrays match, the method proceeds to step 1162. If the signal arrays are not matched, the method restarts at step 1156. Stated in another manner, in the method of FIG. 11, if the signal arrays are not matched, then the heart is again artificially stimulated based on the actual location determined by the first signal array to generate a different, new second signal array. The new second signal array is then superimposed over the first signal array (step 1158), and the signal arrays are compared to see if they match (step 1160), in the same manner as described above.

At step 1162, the actual location of arrhythmogenic foci is confirmed, and the method for determining the location of arrhythmogenic foci in or near the heart is completed.

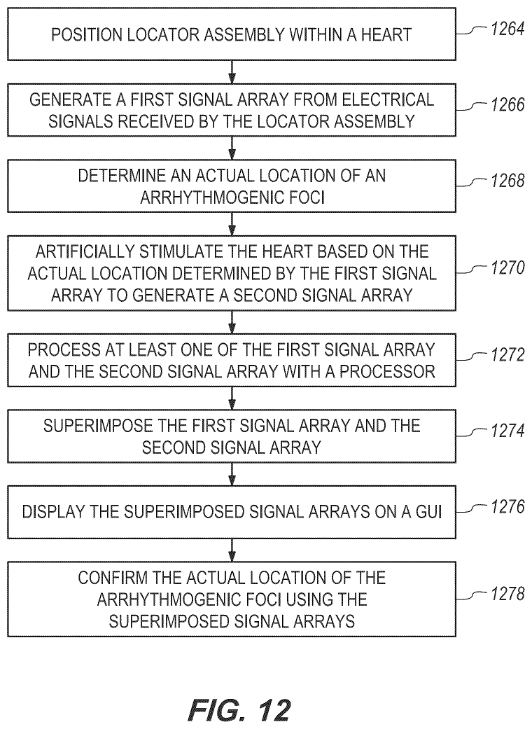

FIG. 12 is a flow chart outlining still another embodiment of a method for determining the location of arrhythmogenic foci in the heart. It is understood that the method pursuant to the disclosure herein can include greater or fewer steps than those shown and described relative to FIG. 12. The method can omit one or more steps illustrated in FIG. 12. The method can add additional steps not shown and described in FIG. 12, and still fall within the purview of the present invention. Further, the sequence of the steps can be varied from those shown and described relative to FIG. 12. The sequence of steps illustrated in FIG. 12 is not intended to limit the sequencing of steps in any manner.

In the embodiment illustrated in FIG. 12, at step 1264, a locator assembly is positioned at any suitable location within the heart, such as in the coronary sinus in one non-exclusive implementation. The locator assembly can include a plurality of electrodes that receive electrical signals from the heart. However, other designs of locator assemblies can be used with the methods described herein.

At step 1266, a first signal array is generated from the electrical signals received by the locator assembly.

At step 1268, an actual location of the arrhythmogenic foci is determined.

At step 1270, the heart is artificially stimulated based on the actual location of the arrhythmogenic foci determined by the first signal array to generate a second signal array. The heart can be artificially stimulated by any suitable device known in the art.

At step 1272, at least one of the first and second signal arrays is processed with a processor.

At step 1274, the first signal array and the second signal array are superimposed.

At step 1276, the superimposed signal arrays are displayed on a graphical user interface. The superimposition of the signal data can be the same and/or similar to the embodiments illustrated in FIGS. 8-9.

At step 1278, the actual location of the arrhythmogenic foci is confirmed using the superimposed signal arrays.

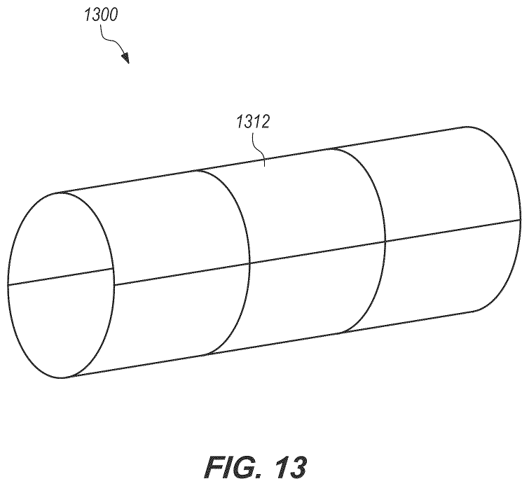

FIG. 13 is a simplified perspective view of still another embodiment of the locator assembly 1300. In this embodiment, the locator assembly 1300 simply includes a device body 1312, such as the self-expanding device body illustrated and described in detail above in relation to FIGS. 6A-6C. In particular, the device body 1312 can include and/or incorporate a spontaneous, passive, self-expanding, lattice-like structural design that is configured to function as a typical stent device in any desired locations within the body of the patient. More specifically, it is noted that the locator assembly 1300 does not include the plurality of electrodes 102 (illustrated in FIG. 1A), the communicator 104 (illustrated in FIG. 1A), the controller 106 (illustrated in FIG. 1A), the routing layer 108 (illustrated in FIG. 1A) and the power source 110 (illustrated in FIG. 1A) as in previous embodiments. Thus, it is appreciated that this embodiment of the locator assembly 1300 would not specifically include the capabilities for locating arrhythmogenic foci 632 (illustrated in FIG. 6D) in or near the heart 101 (illustrated in FIG. 1B), but would rather simply function as a stent for purposes of holding open whatever vessel of the body of the patient in which the locator assembly 1300 is deployed.

It is appreciated that the locator assembly 1300 and/or the device body 1312 can be deployed in any desired locations within the body of the patient using any suitable deployment catheter, such as the deployment catheter 624 illustrated and described above in relation to the embodiment shown in FIGS. 6A-6C. As above, the locator assembly 1300 and/or the device body 1312 will typically be positioned fully within the sheath 623 (illustrated in FIG. 6A) of the deployment catheter 624 (illustrated in FIG. 6A) during initial deployment of the locator assembly 1300. When so positioned fully within the sheath 623, the locator assembly 1300 and/or the device body 1312 will be in the contracted state, as the structure of the sheath 623 will compress the locator assembly 1300 into a more compact configuration, and inhibit the locator assembly 1300 and/or the device body 1312 from moving to the expanded state. Subsequently, the sheath 623 can be moved and/or retracted relative to the locator assembly 1300 so that the locator assembly 1300 and/or the device body 1312 can spontaneously expand to the expanded state, and be positioned at the desired location within the body of the patient. Thus, the locator assembly 1300 and/or the device body 1312 can function solely as a typical stent device at the desired location within the body of the patient.

As noted above, in certain alternative embodiments of the locator assembly, the locator assembly can include the device body with the plurality of electrodes coupled and/or secured thereto, but one or more of the communicator, the controller, the routing layer, and the power source can be positioned away from the device body, such as in a subcutaneous device and/or within an extracorporeal device. In some such embodiments, each of the communicator, the controller, the routing layer, and the power source can be positioned away from the device body, such as in a subcutaneous device and/or within an extracorporeal device. However, it is appreciated that the subcutaneous device and/or the extracorporeal device can include any of the communicator, the controller, the routing layer, and the power source, individually, or in any suitable combination. Certain non-exclusive such embodiments are illustrated and described in greater detail herein below.

FIG. 14 is a simplified illustration of the heart 1401 and yet another embodiment of the locator assembly 1400 having a device body 1412, with one or more components of the locator assembly 1400 being incorporated within a component device 1480 (illustrated as a box) that is spaced apart from the device body 1412. In this embodiment, the locator assembly 1400 is somewhat similar in design and operation to the locator assembly 100 illustrated and described in detail herein above in relation to FIG. 1A. In particular, as shown in FIG. 14, the locator assembly 1400 can again include the device body 1412, and a plurality of electrodes 1402 that are coupled and/or secured to the device body 1412. The device body 1412 and the electrodes 1402 are substantially identical in terms of design and operation to what has been illustrated and described herein above. More specifically, the locator assembly 1400, through use and/or operation of the device body 1412 and the electrodes 1402, is again configured to generate and/or collect information and data (sensing and/or recording electrical signals (such as electrophysiological signals) sent from the heart 1401 and nearby portions of the body) for purposes of locating the arrhythmogenic foci 632 (illustrated in FIG. 6D) in or near the heart 1401.

However, in certain embodiments, one or more of the communicator 1404 (illustrated as a box in phantom), the controller 1406 (illustrated as a box in phantom), the routing layer 1408 (illustrated as a box in phantom), and the power source 1410 (illustrated as a box in phantom) are incorporated within the component device 1480 that is spaced apart from the device body 1412. As illustrated, any of the components of the locator assembly 1400 that are incorporated within the component device 1480, and are thus positioned spaced apart from the device body 1412, can be configured to wirelessly communicate with other components of the locator assembly 1400, such as at least the plurality of electrodes 1402, that are coupled and/or secured to the device body 1412 of the locator assembly 1400.

As shown in FIG. 14, the device body 1412 can again be flexible to conform to portions of the heart 1401 such as valves, veins, sinuses, etc. In particular, in the embodiment shown in FIG. 14, the device body 1412 can again be positioned in a coronary sinus 1427 near a vena cordis media 1428. However, it is understood that the device body 1412 can equally be positioned in other locations in or around the heart 1401, such as in or near a vein of Marshall, or any other suitable location.

In the embodiment specifically illustrated in FIG. 14, each of the communicator 1404, the controller 1406, the routing layer 1408, and the power source 1410 are incorporated within the component device 1480. However, in alternative embodiments, any of the communicator 1404, the controller 1406, the routing layer 1408, and the power source 1410 can still be coupled and/or secured to the device body 1412. It is simply appreciated that in these embodiments of the locator assembly 1400, at least one of the communicator 1404, the controller 1406, the routing layer 1408, and the power source 1410 are incorporated within the component device 1480 that is spaced apart from the device body 1412.

The design and functionality of the communicator 1404, the controller 1406, the routing layer 1408, and the power source 1410 can be substantially similar to what has been illustrated and described in detail herein above. Accordingly, a detailed description of such components will not again be provided in relation to FIG. 14. Moreover, the at least one of the communicator 1404, the controller 1406, the routing layer 1408, and the power source 1410 that are incorporated within the component device 1480 can be configured to wirelessly communicate with the other components of the locator assembly 1400, such as at least the plurality of electrodes 1402, which are coupled and/or secured to the device body 1412. The wireless communication between the components of the locator assembly 1400 that are incorporated within the component device 1480, and the components of the locator assembly 1400, such as the plurality of electrodes 1402, that are coupled and/or secured to the device body 1412, can be accomplished in any suitable manner. More specifically, the component device 1480 and the device body 1412, and the components incorporated therewith, can have any suitable design for purposes of enabling the desired wireless communication between such components, many examples of which have been noted herein above.

The positioning of the component device 1480 can be varied to suit the requirements of the locator assembly 1400. For example, as shown in FIG. 14, in one non-exclusive embodiment, the component device 1480 can be a subcutaneous device that is positioned beneath the skin 1481 of the patient. Alternatively, in another non-exclusive embodiment, the component device 1480 can be an extracorporeal device that is positioned outside of, but near and/or adjacent to, the body of the patient.

As so described, the embodiment of the locator assembly 1400 shown in FIG. 14 can encompass a mapping stent that consists of a cardiac wireless, battery-powered multi-site sensing/recording device. In such embodiment, the controller 1406 includes a control circuit that continuously monitors the patients ECG, and a fibrillation detection algorithm, which when fibrillation is detected, records the pre-fibrillation ECG and the fibrillation data for subsequent transmission to the component device 1480, such as a subcutaneous implantable device. The component device 1480, such as via the controller 1406, can then analyze the recorded ECGs to identify the intra-atrial site initiating the fibrillation. The component device 1480, such as via the communicator 1404, can then transmit the raw ECG data and the analyzed ECG records to an external device 105 (illustrated in FIG. 1) where a physician can download to pre-plan a suitable ablation procedure to treat the atrial fibrillation.

As noted above, in certain embodiments, the locator assembly can be configured to incorporate self-charging capabilities, such that the locator assembly can be recharged and/or self-charged while the locator assembly is positioned within the patient. More specifically, in some embodiments, the power source can encompass and/or include a self-charging design in which the power source effectively charges itself as the locator assembly is performing a diagnostic procedure.

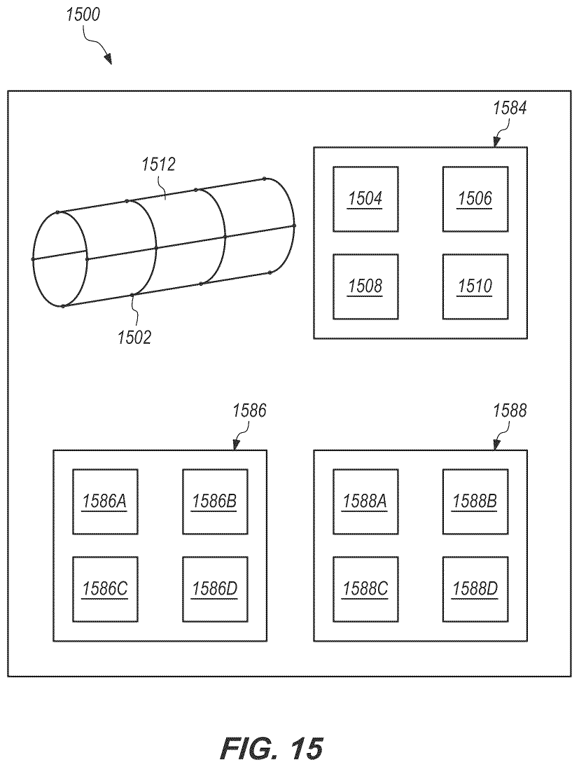

FIG. 15 is a simplified schematic illustration of a portion of still yet another embodiment of the locator assembly 1500. In particular, as illustrated, the locator assembly 1500 again includes a device body 1512 having a plurality of electrodes 1502 coupled and/or secured thereto, and further includes an electronic subsystem 1584 (illustrated as a box), which can include a communicator 1504 (illustrated as a box), a controller 1506 (illustrated as a box), a routing layer 1508 (illustrated as a box) and a power source 1510 (illustrated as a box) that are substantially similar in design and functionality to the corresponding components illustrated and described in detail herein above. It is appreciated that, in different embodiments, each of the noted components of the electronic subsystem 1584 can be coupled and/or secured to the device body 1512 (such as in the embodiment illustrated in FIG. 1A), or can be incorporated within a component device 1480 (such as in the embodiment illustrated in FIG. 14).

However, in this embodiment, in order to facilitate the self-charging capabilities of the locator system 1500, the locator system 1500 further includes an energy harvesting module 1586 (illustrated as a box) that is configured to self-charge electronic components of the locator assembly 1500 as the locator assembly 1500 is being utilized to perform a diagnostic procedure. More particularly, in this embodiment, the locator assembly 1500 can be described as a self-charging intracardiac mapping stent implant, having the device body 1512 configured with electrodes 1502, which can be wirelessly paired with the accommodating electronic subsystem 1584 that can be incorporated, at least in part, within the component device 1480 to provide an autonomous biodata recording and data analysis system. As shown, the locator assembly 1500 can further include the energy harvesting module 1586 and a corresponding analysis module 1588 (illustrated as a box) that cooperate to provide the self-charging capabilities for the electronic subsystem 1584 and/or for the locator assembly 1500 as a whole.

As with the previous embodiments, the device body 1512 is configured to be anchored inside the heart 101 (illustrated in FIG. 1B) and/or its vascular system during the performance of any desired diagnostic procedures.

The energy harvesting module 1586 can have any suitable design for purposes of providing the desired self-charging capabilities to the locator assembly 1500. In many embodiments, the energy harvesting module 1586 can include one or more of an energy storage component 1586A (illustrated as a box), an inertial unit 1586B (illustrated as a box), a translator 1586C (illustrated as a box), and a power management circuit 1586D (illustrated as a box). Alternatively, the energy harvesting module 1586 can have more components or fewer components that what is specifically shown in FIG. 15.

The energy storage component 1586A can have any suitable design for storing the necessary energy or power that is provided to the electronic subsystem 1584 and/or to the locator assembly 1500 as a whole.

During use of the energy harvesting module 1586, the inertial unit 1586B is subject to external stresses which are applied to the device body 1512 under the effect of movements of a wall or inside a vessel to which the device body 1512 is anchored, and/or of blood flow rate variations in the environment surrounding the device body 1512 at the rhythm of heartbeats and/or of cardiac tissue vibrations. The translator 1586C is configured to convert the mechanical energy produced by oscillations of the inertial unit 1586B into an oscillating electrical signal S. The power management circuit 1586D is configured to rectify and regulate the oscillating electrical signal S in order to output a stabilized direct voltage or current for powering the electronic subsystem 1584 and/or for charging the energy storage component 1586A. As such, the energy harvesting module 1586 is configured to convert into electrical energy the external stresses applied to the device body 1512 under the effect of movements of a wall or inside a vessel to which the device body 1512 is anchored and/or of blood flow rate variations in the environment surrounding the device body 1512 at the rhythm of heartbeats and/or of cardiac tissue vibrations.

The analysis module 1588 is configured to receive the oscillating electrical signal S provided by the translator 1586C as an input, and is further configured to analyze variations of the oscillating electrical signal S to derive a value of a physiological parameter of the patient into whom the locator assembly 1500 and/or the device body 1512 has been implanted. The analysis module 1588 can have any suitable design for such purposes. In certain embodiments, the analysis module 1588 can include one or more of a sequencing module 1588A (illustrated as a box), a circuit 1588B (illustrated as a box), a memory module 1588C (illustrated as a box), and a transmission module 1588D (illustrated as a box). Alternatively, the analysis module 1588 can have more components or fewer components than what is specifically shown in FIG. 15.

The sequencing module 1588A initially receives the oscillating electrical signal S from the energy harvesting module 1586. Based on such oscillating electrical signal S, the circuit 1588B is configured to record intracardiac ECGs, and to detect atrial fibrillation and other arrhythmias. The memory module 1588C is configured to save all of the ECG data that has been recorded and/or detected by the circuit 1588B. Subsequently, the transmission module 1588D is configured to wirelessly transfer all of the ECG data to the component device 1480, which, as noted above, can be a subcutaneous device and/or an extracorporeal device.

In summary, as described in detail in various embodiments herein, the present technology provides a system, device, and method for determining the location of arrhythmogenic foci. In certain embodiments, the locator assembly can utilize protective materials such as inner/outer layers and can implement drug elution. The eluted drug is released over time to counteract the pro-thrombotic and inflammatory potential by the inflated locator assembly at its final location. Additionally, the present technology provides a safe housing between the inner and outer layer to host the various elements (integrated circuits, routing layers, power source, antenna) that compose the locator assembly.

Further, in some embodiments, the present technology includes certain components of the locator assembly, such as at least one of the communicator, the controller, the routing layer, and the power source, being incorporated within a component device, such as a subcutaneous device and/or an extracorporeal device in certain embodiments, that is spaced apart from the device body of the locator assembly. In such embodiments, any components of the locator assembly that are incorporated within the component device can still communicate, such as wirelessly, with the plurality of electrodes that are still typically coupled and/or secured to the device body. Such arrangement of communication between the components of the locator assembly enables the locator assembly to function in the intended manner for purposes of determining the location of arrhythmogenic foci.

It is appreciated that the systems, devices, and methods provided herein address multiple potential issues with the performance, reliability, and proper usage of deliverable locator assemblies, in particular locator assemblies that utilize a plurality of bipolar electrodes to determine the location of the focal point of atrial fibrillation. Specific problems solved by the systems, devices, and methods disclosed herein include:
a) The technology disclosed herein improves the deliverable locator technology to enable mapping of precipitating episodes of clinical atrial fibrillation during the patient's daily life;
b) The technology disclosed herein increases the accuracy of the determination of the location of the focal point of atrial fibrillation;
c) The technology disclosed herein reduces the time to determine the location of the focal point of atrial fibrillation;
d) The technology disclosed herein provides recharging, or sometimes self-charging, capabilities for the locator assembly while implanted within the patient; and
e) The technology disclosed herein reduces the risk of thrombus formation and wall bleeding upon delivery and removal of the locator assembly.

It should be noted that, as used in this specification and the appended claims, the singular forms “a,” “an,” and “the” include plural referents unless the content and/or context clearly dictates otherwise. It should also be noted that the term “or” is generally employed in its sense, including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

The headings used herein are provided for consistency with suggestions under 37 CFR 1.77 or otherwise to provide organizational cues. These headings shall not be viewed to limit or characterize the invention(s) set out in any claims that may issue from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the detailed description provided herein. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of systems, devices, and methods for determining the location of arrhythmogenic foci have been illustrated and described herein, one or more features of any one embodiment can be combined with one or more features of one or more of the other embodiments, provided that such combination satisfies the intent of the present invention.

While a number of exemplary aspects and embodiments of the systems, devices, and methods for determining the location of arrhythmogenic foci have been discussed above, those of skill in the art will recognize certain modifications, permutations, additions, and sub-combinations thereof. It is therefore intended that the following appended claims and claims hereafter introduced are interpreted to include all such modifications, permutations, additions, and sub-combinations as are within their true spirit and scope, and no limitations are intended to the details of construction or design herein shown.

What is claimed is:

1. A locator assembly for determining a location of arrhythmogenic foci in or near a heart within a body of a patient, the locator assembly comprising:
a device body provided in the form of an expandable stent that is configured to be inserted into and engage the heart;
a plurality of electrodes that are coupled to the device body, the plurality of electrodes being configured to sense electrical signals from the heart to determine the location of the arrhythmogenic foci within the body of the patient;
a component device that is positioned spaced apart from the device body; and
at least one of (i) a communicator that is configured to receive data regarding the sensed electrical signals from the plurality of electrodes and to transmit the data to an external device, (ii) a controller that is configured to control operation of the plurality of electrodes, and (iii) a power source that is configured to provide power to the plurality of electrodes, that is incorporated within the component device, the at least one of the communicator, the controller, and the power source being configured to wirelessly communicate with the plurality of electrodes.

2. The locator assembly of claim 1 further comprising at least two of the communicator, the controller, and the power source that are incorporated within the component device, the at least two of the communicator, the controller, and the power source being configured to wirelessly communicate with the plurality of electrodes.

3. The locator assembly of claim 2 further comprising each of the communicator, the controller, and the power source that are incorporated within the component device, each of the communicator, the controller, and the power source being configured to wirelessly communicate with the plurality of electrodes.

4. The locator assembly of claim 1 wherein the component device is a subcutaneous device that is positioned under skin of the patient.

5. The locator assembly of claim 1 wherein the component device is an extracorporeal device that is positioned adjacent to, but outside of the body of the patient.

6. The locator assembly of claim 1 further comprising a routing layer that interconnects the plurality of electrodes.

7. The locator assembly of claim 1 further comprising the power source that provides power to the plurality of electrodes.

8. The locator assembly of claim 7 wherein the power source is rechargeable.

9. The locator assembly of claim 7 wherein the power source is self-charging.

10. The locator assembly of claim 9 further comprising an energy harvesting module that enables the power source to be self-charging, the energy harvesting module including (i) an inertial unit that is subject to external stresses that are applied to the device body when positioned inside the body of the patient, the external stresses causing oscillations of the inertial unit, and (ii) a translator that is configured to convert mechanical energy produced by the oscillations of the inertial unit into an oscillating electrical signal.

11. The locator assembly of claim 10 wherein the energy harvesting module further includes a power management circuit and an energy storage component, the power management circuit being configured to regulate the oscillating electrical signal in order to output a stabilized direct voltage or current for at least one of powering the plurality of electrodes and charging the energy storage component.

12. The locator assembly of claim 1 wherein the device body is provided in the form of a self-expanding stent that is configured to be inserted into and engage the heart.

13. The locator assembly of claim 12 wherein the device body is configured to spontaneously move from a contracted state wherein the device body has a contracted diameter, to an expanded state wherein the device body has an expanded diameter that is greater than the contracted diameter.

14. The locator assembly of claim 13 wherein a ratio of the expanded diameter to the contracted diameter is less than 20:1 and greater than 1:1.

15. The locator assembly of claim 1 wherein at least two of the plurality of electrodes are positioned circumferentially about the device body; and wherein at least two of the plurality of electrodes are positioned longitudinally along the device body.

16. The locator assembly of claim 1 wherein the plurality of electrodes includes a plurality of anodes and cathodes that form a plurality of bipoles.

17. The locator assembly of claim 1 wherein the plurality of electrodes includes an electrocardiogram electrode.

18. A locator system comprising a deployment catheter including a sheath; and the locator assembly of claim 1; wherein the device body is configured to spontaneously move from a contracted state to an expanded state; and wherein the device body is positioned within the sheath when the device body is inserted into the heart, the sheath being configured to maintain the device body in the contracted state.

19. The locator system of claim 18 wherein the device body spontaneously moves from the contracted state to the expanded state when the device body is removed from the sheath.

20. A method for determining a location of arrhythmogenic foci in or near a heart within a body of a patient, the method comprising the steps of:
coupling a plurality of electrodes to a device body to form at least a portion of a locator assembly, the device body including an expandable stent;
inserting the device body within the heart;
sensing electrical signals from the heart with the plurality of electrodes of the locator assembly;
positioning a component device spaced apart from the device body;
incorporating at least one of (i) a communicator that receives data regarding the sensed electrical signals from the plurality of electrodes and transmits the data to an external device, (ii) a controller that controls operation of the plurality of electrodes, and (iii) a power source that provides power to the plurality of electrodes, within the component device;
wirelessly coupling the at least one of the communicator, the controller, and the power source with the plurality of electrodes; and
determining the location of the arrhythmogenic foci within the body of the patient based at least in part on the electrical signals received from the heart by the plurality of electrodes.
